(19) United States
(12) Patent Application Publication
(10) Pub. No.: US 2026/0232500 A1
(43) Pub. Date: Aug. 13, 2026

(54) ABSORBENT ARTICLE WITH ABSORBENT CORE STRUCTURE HAVING A SHAPED INNER CORE LAYER

(71) Applicant: The Procter & Gamble Company, Cincinnati, OH (US)

(72) Inventors: Christopher Philip BEWICK-SONNTAG, Cincinnati, OH (US); John David NORCOM, Cincinnati, OH (US); Clint Adam MORROW, Union, KY (US); Ashley Nicole BASIUS, Cincinnati, OH (US); Amber Dae BERTHOLD, Brookville, IN (US)

(21) Appl. No.: 19/531,816

(22) Filed: Feb. 6, 2026

Related U.S. Application Data
(60) Provisional application No. 63/755,456, filed on Feb. 7, 2025.

(51) Int. Cl. A61F 13/535; A61F 13/15; A61F 13/472; A61F 13/53

(52) U.S. Cl. CPC A61F 13/535; A61F 13/53; A61F 2013/15373; A61F 2013/15406; A61F 2013/15447; A61F 13/472; A61F 2013/530226; A61F 2013/530343; A61F 2013/530489

(57) ABSTRACT

A disposable absorbent article includes an absorbent core structure disposed between a topsheet and a backsheet. The absorbent core structure may include an upper nonwoven layer; a lower nonwoven layer; and an inner core layer disposed between the upper nonwoven layer and the lower nonwoven layer. The inner core layer may include a first absorbent zone having a first basis weight and a second absorbent zone having a second basis weight, wherein the first basis weight is greater than the second basis weight. The first absorbent zone may include two lateral side zones and a central zone, wherein an outer portion of the central zone is positioned inward from an outer portion of the two lateral side zones, and wherein at least a portion of the central zone is continuous with at least a portion of the two lateral side zones. The inner core layer may be a unitary structure.

### CROSS REFERENCE TO RELATED APPLICATION

This application claims the benefit, under 35 USC 119(e), to U.S. Provisional Patent Application No. 63/755,456, filed on Feb. 7, 2025, which is herein incorporated by reference in its entirety.

### FIELD OF THE INVENTION

The present disclosure relates to absorbent articles comprising an absorbent core structure having a shaped inner core layer.

### BACKGROUND OF THE INVENTION

Absorbent articles are widely used among consumers, e.g., diapers, training pants, feminine pads, adult incontinence pads, etc. Generally, absorbent articles such as these comprise a topsheet and a backsheet, with an absorbent core structure disposed therebetween. These absorbent articles are designed to absorb and retain liquids and other discharges from the human body to prevent body and garment soiling. To absorb fluid effectively without leakage, absorbent articles for menstrual applications should conform closely to a women's genital anatomical shape such that the absorbent article captures fluid as it exits the labial structure. A common complaint from users of current absorbent articles is the sensation of fluid moving along the body or escaping the article during heavier discharge events. In the case of undergarment-applied products, the undergarment, also referred to as a panty, and absorbent article often do not fit sufficiently close to the body to address these consumer concerns. Historically, absorbent articles have tried to address this problem by uniformly adding more overall absorbent material thickness to attempt to fill the space between the user's legs. Other approaches have added more absorbent material to the center of the absorbent article (commonly referred to as “more in the middle”) in order to increase the caliper in the central region. This is often accomplished by profiling the cellulose absorbent material in the longitudinal direction or by adding an additional, discrete ellipsoid-shaped absorbent layer.

However, these traditional approaches do not adequately solve the problem of reducing or eliminating the spread of fluid on the body because the additional absorbent material is invariably bulky, stiff, and not shaped to closely and comfortably conform to the wearer's intimate anatomy. In the case of female genitalia, fluid first exits the body internally within the labial vestibule, in particular within the labial minora, prior to exiting through the labial majora. Fluid can exit the labial structure at the top, the bottom, and/or at the sides. Since the labial vestibule is curved, there typically exists a gap between the labia majora such that fluid exiting the labial minora is not captured by simply adding more absorbent material in the central region. Traditional “more in the middle” shapes rest atop of the labial structure and thus forms a bridge across the gap between the labial majora without actually capturing the fluid that resides and is moving from within the labial vestibule outwards. Additionally, typical bulky central regions may actually push the non-raised portions of the absorbent article away from the body, thus causing more gapping on the sides for fluid to spread and be felt by the consumer.

Thus, there is a need for an improved absorbent article that more effectively captures fluid as it exits the labial minora, within the labia majora, across the diverse range of female genitalia shapes and sizes without being stiff and bulky.

### SUMMARY OF THE INVENTION

The present disclosure solves the problem of fluid spreading on the body by providing an absorbent core structure comprising an inner core layer that is shaped to at least partially fit around the sides of the labial majora and to at least partially fit into the perineum. As described herein, the absorbent article comprises an absorbent core structure comprising a sculptural inner core layer which is highly compressible and moldable without being bulky, allowing the absorbent article to comfortably adapt and fit closely to a wide range of female anatomical shapes.

In some embodiments, a disposable absorbent article comprises a topsheet; a backsheet; and an absorbent core structure disposed between the topsheet and the backsheet, wherein the absorbent core structure comprises: an upper nonwoven layer; a lower nonwoven layer; and an inner core layer disposed between the upper nonwoven layer and the lower nonwoven layer, wherein the inner core layer comprises cellulosic fibers; wherein the inner core layer comprises a first absorbent zone having a first basis weight and a second absorbent zone having a second basis weight, the first basis weight is greater than the second basis weight; and wherein the first absorbent zone comprises two lateral side zones and a central zone, wherein an outer portion of the central zone is positioned inward from an outer portion of the two lateral side zones, and wherein at least a portion of the central zone is continuous with at least a portion of the two lateral side zones; wherein the inner core layer is a unitary structure.

In some embodiments, a disposable absorbent article comprising: a topsheet; a backsheet; and an absorbent core structure disposed between the topsheet and the backsheet, wherein the absorbent core structure comprises: an upper nonwoven layer; a lower nonwoven layer; and an inner core layer disposed between the upper nonwoven layer and the lower nonwoven layer, wherein the inner core layer comprises cellulosic fibers and superabsorbent particles; wherein the inner core layer comprises a first absorbent zone having a first basis weight and a second absorbent zone having a second basis weight, wherein the first absorbent zone comprises two lateral side zones and the first basis weight is greater than the second basis weight; wherein the absorbent article has a first average density measured in the first absorbent zone and a second average density measured in the second absorbent zone, wherein at least a portion the central zone is continuous with at least a portion of the two lateral side zones, and wherein the second average density is within about 0% to about 20% of the first density.

### BRIEF DESCRIPTION OF THE DRAWINGS

FIG. 1 is a plan view of an example absorbent article, wearer-facing surface facing the viewer;

FIG. 2A-B are cross-sectional views of the absorbent article of FIG. 1 taken along line 2-2;

FIG. 3 is a plan view of an example absorbent article, wearer-facing surface facing the viewer, illustrating an exemplary tapered inner core layer shape, according to one or more configurations shown and described herein;

FIG. 4 is a plan view of an example absorbent article, wearer-facing surface facing the viewer, according to one or more configurations shown and described herein;

FIG. 5A-B is a plan view of an example absorbent article, wearer-facing surface facing the viewer, illustrating first absorbent zone and second absorbent zone configurations, according to one or more configurations shown and described herein;

FIG. 5C-D is a close up illustration of a first absorbent zone, according to one or more configurations shown and described herein;

FIG. 6A-C is a plan view of an example absorbent article, wearer-facing surface facing the viewer, illustrating first absorbent zone and second absorbent zone configurations, according to one or more configurations shown and described herein;

FIG. 7A is a cross-sectional view of the absorbent article of FIG. 5A taken along line 7A-7A, illustrating the profile of the inner core layer in the absorbent article, according to one or more configurations shown and described herein;

FIG. 7B is a cross-sectional view of the absorbent article of FIG. 5A taken along line 7B-7B, illustrating the profile of the inner core layer in the absorbent article, according to one or more configurations shown and described herein;

FIG. 8A-D are plan views of example absorbent articles, wearer-facing surface facing the viewer, illustrating first absorbent zone and second absorbent zone configurations, according to one or more configurations shown and described herein;

FIG. 9 is a plan view of an example absorbent article, wearer-facing surface facing the viewer, illustrating first absorbent zone and second absorbent zone configurations, according to one or more configurations shown and described herein;

FIG. 10A is a close up illustration of a structural bond site, according to one or more configurations shown and described herein;

FIG. 10B is a cross-sectional view of the structural bond site of FIG. 10A;

FIG. 11 is a plan view of an example absorbent article, wearer-facing surface facing the viewer, illustrating flex bond channel regions, according to one or more configurations shown and described herein;

FIG. 12 is a plan view of an example absorbent article, wearer-facing surface facing the viewer, according to one or more configurations shown and described herein;

FIG. 13 is a plan view of an example absorbent article, wearer-facing surface facing the viewer, according to one or more configurations shown and described herein;

FIG. 14 is a plan view of an example absorbent article, wearer-facing surface facing the viewer, according to one or more configurations shown and described herein;

FIG. 15A-C are a test method arrangement for the Dry CD Ultra Sensitive 3 Point Bending Method; and

FIG. 16 is a two-dimensional line profile for the Flex Bond Channel Measurements.

### DETAILED DESCRIPTION OF THE INVENTION

As used herein “disposable absorbent article” or “absorbent article” shall be used in reference to articles such as diapers, training pants, diaper pants, refastenable pants, adult incontinence pads, adult incontinence pants, feminine hygiene pads, cleaning pads, and the like, each of which are intended to be discarded after use.

As used herein “absorbent core structure” shall be used in reference to the upper nonwoven layer, the lower nonwoven layer, and the inner core layer disposed between the upper nonwoven layer and the lower nonwoven layer. As used herein, “absorbent core structure” does not include any secondary topsheet, topsheet, secondary backsheet, or backsheet of the absorbent article.

As used herein “hydrophilic” and “hydrophobic” have meanings as well established in the art with respect to the contact angle of water on the surface of a material. Thus, a material having a water contact angle of greater than about 90 degrees is considered hydrophobic, and a material having a water contact angle of less than about 90 degrees is considered hydrophilic. Compositions which are hydrophobic, will increase the contact angle of water on the surface of a material while compositions which are hydrophilic will decrease the contact angle of water on the surface of a material. Notwithstanding the foregoing, reference to relative hydrophobicity or hydrophilicity between a material and a composition, between two materials, and/or between two compositions, does not imply that the materials or compositions are hydrophobic or hydrophilic. For example, a composition may be more hydrophobic than a material. In such a case neither the composition nor the material may be hydrophobic; however, the contact angle exhibited by the composition is greater than that of the material. As another example, a composition may be more hydrophilic than a material. In such a case, neither the composition nor the material may be hydrophilic; however, the contact angle exhibited by the composition may be less than that exhibited by the material.

As used herein, the term “filament” refers to any type of continuous strand produced through a spinning process, a meltblowing process, a melt fibrillation or film fibrillation process, or an electrospinning production process, or any other suitable process to make filaments. The term “continuous” within the context of filaments are distinguishable from staple length fibers in that staple length fibers are cut to a specific target length. In contrast, “continuous filaments” are not cut to a predetermined length, instead, they can break at random lengths but are usually much longer than staple length fibers.

As used herein, “machine direction” refers to the direction in which a web flows through an absorbent article converting process. For the sake of brevity, “machine direction” may be referred to as “MD”.

As used herein, “cross machine direction” refers to the direction which is perpendicular to the MD. For the sake of brevity, “cross machine direction” may be referred to as “CD”.

“Decitex” also known as Dtex is a measurement used in the textile industry used for measuring yarns or filaments. 1 Decitex=1 gram per 10,000 meters. In other words, if 10,000 linear meters of a yarn or filament weights 500 grams that yarn or filament would have a decitex of 500.

As used herein, “resilient” refers to a material that tends to retain its shape both in the dry and wet states and when subjected to a compression or tensile force tends to recover its original, pre-compression or pre-elongated shape when such force is removed. In some aspects, the upper and/or lower nonwoven layers described herein may be resilient.

As used herein, “wearer-facing” (sometimes referred to herein as body-facing) and “outward-facing” (sometimes referred to herein as garment-facing) refer respectively to the relative location of an element or a surface of an element or group of elements. “Wearer-facing” implies the element or surface is nearer to the wearer during wear than some other element or surface. “Outward-facing” implies the element or surface is more remote from the wearer during wear than some other element or surface (i.e., element or surface is proximate to the wearer's garments that may be worn over the absorbent article).

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

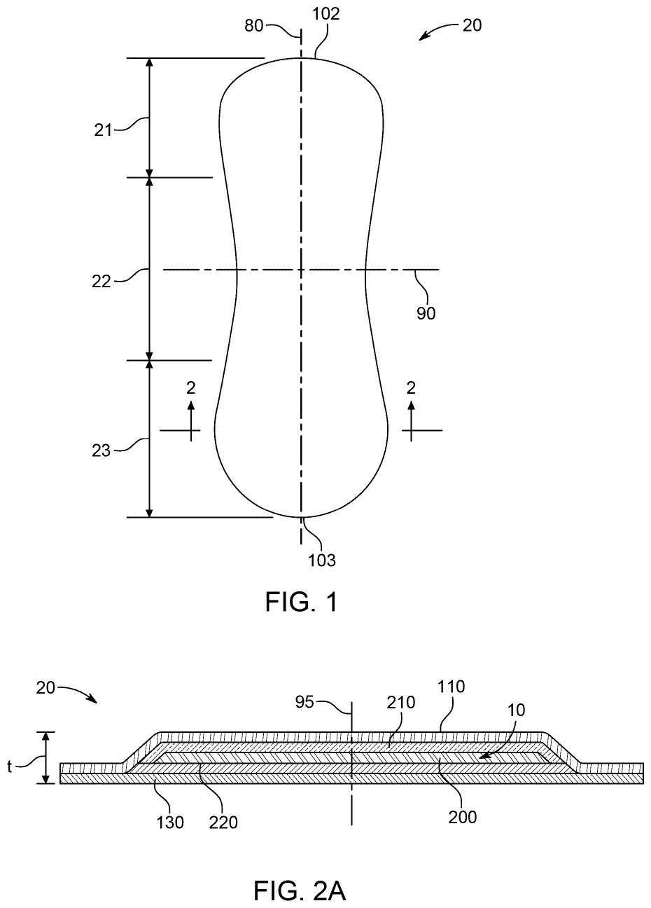

The present disclosure relates to disposable absorbent articles. An exemplary absorbent article 20 of the present disclosure is represented in FIG. 1. The absorbent article 20 of the present disclosure is designed to be resilient and conformable and deliver a superior in-use experience without substantially bunching and/or compressing, which will be discussed in more detail herein. The absorbent article 20 of the present disclosure is designed to recover to its original state when exposed to bodily forces. The absorbent article 20 of FIG. 1 is shown with a longitudinal axis 80, a lateral axis 90, a front edge 102, and a rear edge 103. The absorbent article 20 has a front region 21, a middle region 22, and a rear region 23. The absorbent article 20 may be symmetric about the longitudinal axis 80 or asymmetric about the longitudinal axis 80. Similarly, the absorbent article 20 may be symmetric about the lateral axis 90 or asymmetric about the lateral axis 90.

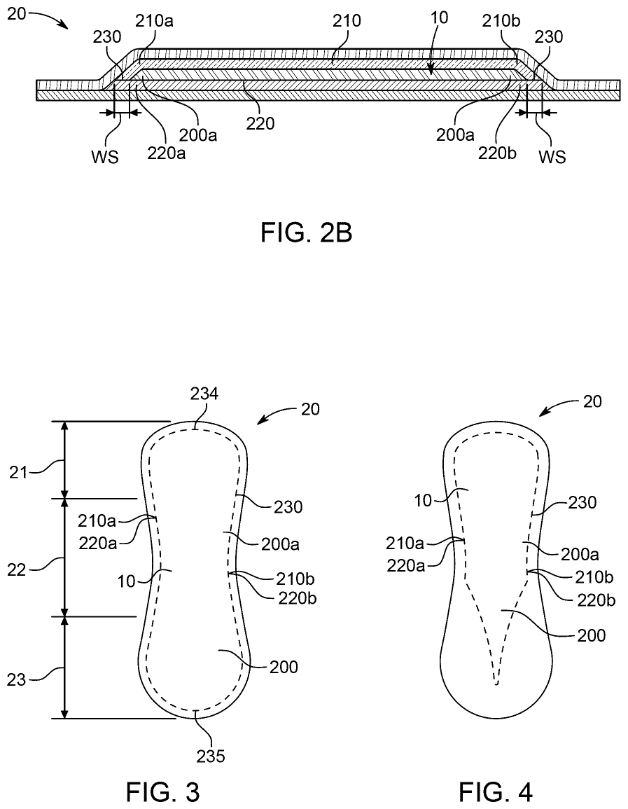

FIG. 2A represents a cross section of FIG. 1 taken along the line 2. Referring to FIG. 2A, the absorbent article 20 may comprise a topsheet 110 and a backsheet 130 opposite the topsheet 110. Topsheet 110 may be formed of any suitable nonwoven web or formed film material. The topsheet 110 is positioned adjacent a wearer-facing surface of the absorbent article 20 and may be joined to the backsheet 130 by any suitable attachment or bonding method. At least a portion of each of the topsheet 110 and the backsheet 130 may be joined directly to each other in the peripheral regions outside the perimeter of the absorbent core structure and/or may be indirectly joined by directly joining them respectively to wearer-facing and outward-facing surfaces of the absorbent article or additional optional layers included with the absorbent article.

The absorbent article 20 may have any known or otherwise effective topsheet 110, such as one which is compliant, soft feeling, and non-irritating to the wearer's skin. A suitable topsheet material will include a liquid pervious material that is comfortable when in contact with the wearer's skin and permits discharged menstrual fluid to rapidly penetrate through it. Some suitable examples of topsheet materials include films, nonwovens, laminate structures including film/nonwoven layers, film/film layers, and nonwoven/nonwoven layers.

In order to ensure that fluid contacting the top (wearer-facing) surface of a topsheet will move suitably rapidly in a z-axis direction to the bottom (outward-facing) surface of the topsheet where it can be drawn into the absorbent article, the topsheet web material such as a nonwoven forming the topsheet should have an appropriate weight/volume density, reflecting suitable presence of interstitial passageways (sometimes known as “pores”) among and between the constituent fibers, through which fluid may move within the nonwoven material. In some circumstances a nonwoven material topsheet with fibers that are consolidated too densely may have insufficient numbers and/or volumes and/or sizes of pores, and the nonwoven will obstruct rather than facilitate rapid downward z-direction fluid movement. On the other hand, a nonwoven with fibers that are too large and/or not consolidated enough to provide a certain level of opacity (for purposes of concealing absorbed fluid in the layers beneath) and a substantial appearance may be negatively perceived by users. To balance competing needs for opacity and loft (which call for a higher caliper) vs. a limitation on the z-axis direction distance that discharged fluid travels through the topsheet from the wearer-facing surface to the outward-facing surface to reach the absorbent core structure below, the caliper of the topsheet material may be controlled. The caliper is controlled by the adjustment of pore size, pore volume, and number of pores via selection of appropriate fiber size, basis weight, and extent of consolidation. Through optimization of these parameters, it has been found that a desirable topsheet has a caliper of from about 0.20 mm to about 1.0 mm, or from about 0.25 mm to about 0.80 mm, or from about 0.30 mm to about 0.60 mm, specifically including all values within these ranges and any ranges created thereby.

In combination with adjustment of pore size, pore volume, and number of pores via selection of appropriate fiber size, basis weight, and extent of consolidation, the topsheet may include fiber constituents for having particular surface chemistry(ies), e.g., fibers with hydrophobic surfaces, hydrophilic surfaces, or a blend of differing fibers and/or z-direction stratification or gradient thereof. Fibers having hydrophilic surfaces will tend to attract and move aqueous components of menstrual fluid there along in a manner conducive to wicking and rapid fluid acquisition following discharge. At the same time, however, a predominance of hydrophilic fiber surfaces within the topsheet may increase a tendency of the topsheet to reacquire fluid from absorbent components beneath (rewet), which can cause an undesirable wet feel for the user. On the other hand, fibers having hydrophobic surfaces will tend to repel aqueous components of menstrual fluid and/or resist movement of fluid along their surfaces, thereby tending to resist wicking—but also to resist rewetting. The manufacturer may wish to seek an appropriate balance in selecting constituent fibers having hydrophilic surfaces, fibers having hydrophobic surfaces, or a blend and/or z-direction stratification thereof, in combination with fiber size, fiber consolidation level, and resulting topsheet pore size, volume and number, for any particular product design.

The topsheet 110 may be liquid pervious permitting liquids (e.g., urine, menses) to readily penetrate through its thickness. Some suitable examples of topsheet materials include films, nonwovens, laminate structures including film/nonwoven layers, film/film layers, and nonwoven/nonwoven layers. Other exemplary topsheet materials and designs are disclosed in U.S. Patent Application Publication Nos. 2016/0129661, 2016/0167334, and 2016/0278986.

Nonlimiting examples of nonwoven web materials that may be suitable for use to form the topsheet 110 include fibrous materials made from natural fibers, modified natural fibers, synthetic fibers, or combinations thereof “Natural fibers”, as used herein, means fibers that can be obtained from animals, plants, or minerals. Natural fibers may include cellulosic materials, such as pine, fir, spruce, hemlock, larch, maple, oak, elm, birch, poplar, aspen, eucalyptus, cotton, flax, bamboo, wheat straw, red algae and/or other seaweeds, and hemp fibers. “Modified natural fibers” refers to natural fibers that have undergone additional processing and may include fibers obtained from animals, plants, or minerals that have undergone modification, such as rayon and viscose. Some suitable examples are described in U.S. Pat. Nos. 4,950,264; 4,988,344; 4,988,345; 3,978,185; 7,785,690; 7,838,099; 5,792,404; and 5,665,452.

The topsheet 110 may be formed of a nonwoven web material of a spunbond web including single-component continuous fibers, bi-component or multi-component fibers, or a blend of single-component fibers spun of differing polymer resins, or any combination thereof. The topsheet may also be a formed nonwoven topsheet as disclosed in US Patent Publication No. 2019/0380887.

The topsheet 110 may include tufts as described in U.S. Pat. Nos. 8,728,049; 7,553,532; 7,172,801; 8,440,286; 7,648,752; and 7,410,683. The topsheet 110 may have a pattern of discrete hair-like fibrils as described in U.S. Pat. No. 7,655,176 or U.S. Pat. No. 7,402,723. Additional examples of suitable topsheet materials include those described in U.S. Pat. Nos. 8,614,365; 8,704,036; 6,025,535; and US Patent Publication No. 2015/041640. Another suitable topsheet may be formed from a three-dimensional substrate as detailed in US 2017/0258647. The topsheet may have one or more layers, as described in US Patent Publication Nos. 2016/0167334; 2016/0166443; and 2017/0258651.

The backsheet 130 may be positioned opposite the topsheet 110, such as illustrated in FIG. 2A. The backsheet 130 may be positioned beneath or subjacent an outward-facing surface of the absorbent core structure 10 (discussed in more detail hereinbelow) and may be joined thereto by any suitable attachment methods. For example, the backsheet 130 may be secured to the absorbent core structure 10 by a uniform continuous layer of adhesive, a patterned layer of adhesive, or an array of separate lines, spirals, or spots of adhesive. Alternatively, the attachment method may include heat bonds, pressure bonds, ultrasonic bonds, dynamic mechanical bonds, or any other suitable attachment mechanisms or combinations thereof. The absorbent core structure 10 may be joined directly to the backsheet 130. Alternatively, the absorbent core structure 10 may not be joined directly to the backsheet 130, but may be separated by one or more intervening layers.

The backsheet 130 may be impermeable or substantially impermeable by aqueous liquids (e.g., urine, menstrual fluid). The backsheet 130 may be manufactured from thin plastic film or other flexible liquid impermeable materials that are substantially free of synthetic material. As used herein, the term “flexible” refers to materials which are compliant and will readily conform to the general shape and contours of the human body. The backsheet 130 may prevent, or at least substantially inhibit, fluids absorbed and contained within the absorbent core structure 10 from escaping and reaching articles of the wearer's clothing that contact the absorbent article 20, such as undergarments and outer clothing. However, in some instances, the backsheet 130 may be made and/or adapted to permit vapor to escape from the absorbent core structure 10 (i.e., the backsheet is made to be breathable), while in other instances the backsheet 130 may be made so as not to permit vapors to escape (i.e., it is made to be non-breathable). Thus, the backsheet 130 may comprise a polymeric film such as thermoplastic films of polyethylene and/or polypropylene. A suitable material for the backsheet 130 is a thermoplastic film having a thickness of from about 0.012 mm (0.5 mil) to about 0.051 mm (2.0 mils), specifically including all values within these ranges and any ranges created thereby. Any suitable backsheet known in the art may be utilized with the present invention.

The backsheet 130 may be a single layer or multi-layered (more than one layer). For example, a backsheet 130 having at least two layers may include: a first layer comprising a vapor permeable aperture-formed film layer and a second layer comprising a breathable microporous film layer, as described in U.S. Pat. No. 6,462,251. Other suitable examples of dual or multi-layer breathable backsheets for use herein include those described in U.S. Pat. Nos. 3,881,489; 4,341,216; 4,713,068; 4,818,600; EP 203 821; EP 710 471; EP 710 472; and EP 0 793 952. Suitable single layer breathable backsheets for use herein include those described for example in GB A 2184 389; GB A 2184 390; GB A 2184 391; U.S. Pat. Nos. 4,591,523; 3,989,867; 3,156,242; WO 97/24097; U.S. Pat. Nos. 6,623,464; 6,664,439; and 6,436,508. Some suitable examples of materials for forming a backsheet are described in U.S. Pat. Nos. 5,885,265; 4,342,314; and 4,463,045.

Referring still to FIG. 2A, the absorbent article 20 may further include an absorbent core structure 10 disposed between the topsheet 110 and the backsheet 130. The absorbent core structure 10 may comprise an upper nonwoven layer 210 and a lower nonwoven layer 220 (also referred to herein collectively as upper and lower nonwoven layers or upper and lower nonwovens) and an inner core layer 200 disposed between the upper nonwoven layer 210 and the lower nonwoven layer 220.

As illustrated in FIG. 2A, the absorbent article 20 has a thickness, t, extending from the topsheet 110 to the backsheet 130 along a z-axis 95. The thickness, t, may be uniform or non-uniform across the absorbent article 20. To maintain consumer comfort, the absorbent core 10 may be configured to recover or substantially recover the thickness upon being compressed. Stated another way, the absorbent core structure 10 may be configured to compress and recover its original shape (dry or wet) across a range of bodily movements and compressions. The flexibility and/or resiliency of the absorbent core structure results in an absorbent article that comfortably conforms to the wearer's anatomical geometry while efficiently managing the fluid as it exits the body. This can, unexpectedly, be achieved without typical densification stiffening (for wet integrity) by leveraging resilient upper and lower nonwoven layers composed of resilient polymers located above and below the loosely packed absorbent material of the inner core layer. The absorbent core structure is surprisingly able to carry the structural load and recover shape without physically being stiff or losing the desired structural properties when the absorbent core structure becomes wet. Without being limited by theory, it is believed that wet integrity/shape stability in a cellulose rich absorbent core structure without substantial densification and stiffening may be achieved when select resilient upper and lower nonwovens are positioned above and below the absorbent material of the inner core layer and are joined to and around the absorbent material. The upper and lower nonwovens may have sufficient recovery force to carry the absorbent material back to the original state and/or a stable fiber orientation state following compression by bodily forces. Wrapping or encapsulating a cellulose rich fluff core with a simple cellulose tissue or less resilient nonwoven material may not exhibit sufficient recovery energy to recover shape in-use and particularly when wetted. Structural, wet resilient nonwovens detailed herein may exhibit recovery energies following compression that are sufficient to recover the cellulose rich fiber matrix and are chosen to deliver high compression recovery, with relatively low stiffness, in both dry and wet states. It is believed that suitable absorbent core structures have a low force to compress (less resistance) and the structure is able to recover its shape as the user, in a cyclic fashion, compresses and releases the compressive force with various body movements. To achieve this, the structure should sustain sufficient recovery energy following multiple cyclic compressions. Without sufficient recovery energy, the structure will remain in a compressed bunched state with insufficient force (stored energy) to recover.

Referring to FIG. 2B, the topsheet 110 may be in direct contact with the upper nonwoven layer 210, the upper nonwoven layer 210 may be in direct contact with the inner core layer 200, and/or the inner core layer 200 may be in direct contact with the lower nonwoven layer 220. By “direct contact”, it is meant that there is no further intermediate component layer between the respective layer in direct contact thereto. It is however not excluded that an adhesive material may be disposed between at least a portion of the layers described above. Thus, a first layer may be in direct contact with a second layer when an adhesive material is present between the first layer and the second layer.

Suitable upper nonwoven layers 210 may have a basis weight of from about 30 gsm to about 85 gsm, or from about 35 gsm to about 70 gsm, or from about 40 gsm to about 60 gsm specifically including all values within these ranges and any ranges created thereby. The upper nonwoven layer may have a Tensile Stiffness of from about 0.1 N/mm to about 2.2 N/mm, or from about 0.3 N/mm to about 1.6 N/mm specifically including all values within these ranges and any ranges created thereby as measured according to the CD Cyclic Elongation to 3% Strain Method. The upper nonwoven layer may have a Strain to Break of greater than about 10%, or from about 10% to about 50%, or from about 20% to about 40%, specifically including all values within these ranges and any ranges created thereby as measured according to the Strain to Break Method. The upper nonwoven layer may have a Permanent Strain of about 0.005 to about 0.013 mm/mm, or from 0.005 to about 0.0090 mm/mm, specifically including all values within these ranges and any ranges created thereby as measured according to the CD Cyclic Elongation to 3% Strain Method. The upper nonwoven layer may have Thickness at 7 g/cm² pressure of from about 0.3 mm to about 1.3 mm and/or a Thickness at 70 g/cm² pressure of from about 0.2 mm to about 0.7 mm specifically including all values within these ranges and any ranges created thereby, as measured according to the Nonwoven Thickness—Pressure Method.

The upper nonwoven layer 210 may comprise fibers having a staple length of greater than about 10 mm, or greater than about 25 mm, or from about 10 mm to about 100 mm, or from about 20 mm to about 75 mm, or from about 25 mm to about 50 mm, specifically including all values within these ranges and any ranges created thereby. The upper nonwoven layer 210 may comprise fibers having a fiber diameter of from about 1.3 DTex to about 10.0 DTex, alternatively from about 1.3 DTex to about 6.0 DTex, alternatively from about 2.0 DTex to about 5.0 DTex, specifically including all values within these ranges and any ranges created thereby. Without being limited by theory, it is believed that if the fibers of the upper nonwoven layer are less than about 1.3 Dtex, there may be insufficient air flow through the material during manufacturing.

The upper nonwoven layer 210 may comprise a blend of staple fibers. When the upper nonwoven layer 210 comprises a blend of staple fibers, the blend of fibers preferably comprises 30% or less of fibers having a fiber diameter of 1.3 Dtex and/or 30% or less of fibers having a fiber diameter of 10.0 Dtex. The upper nonwoven layer may comprise fibers, wherein the fibers are a blend of staple fibers having an average fiber diameter of from about 2.0 DTex to about 8.0 DTex, specifically including all values within these ranges and any ranges created thereby. Without being limited by theory, it is believed that fibers having an average fiber diameter of from about 2.0 Dtex to about 8.0 Dtex will help to enable sufficient air flow through the material during manufacturing of the absorbent core structure.

The lower nonwoven layer 220 may comprise fibers having a length of greater than about 10 mm, or greater than about 25 mm, or from about 10 mm to about 100 mm, or from about 20 mm to about 75 mm, or from about 25 mm to about 50 mm, specifically including all values within these ranges and any ranges created thereby. The lower nonwoven layer 220 may comprise continuous fibers. The lower nonwoven layer 220 may comprise fibers having a fiber diameter of from about 1.3 DTex to about 5.0 DTex, or from about 1.3 DTex to about 3.3 DTex, or from about 1.3 DTex to about 2.2 DTex, or from about 2.0 DTex to about 10.0 DTex, specifically including all values within these ranges and any ranges created thereby. The lower nonwoven layer 220 may comprise fibers, wherein the fibers are a blend of fibers having a fiber diameter of from about 0.1 DTex to about 6.0 DTex, specifically including all values within these ranges and any ranges created thereby.

Suitable lower nonwoven layers 220 may have a basis weight of from about 7 gsm to about 40 gsm, or from about 15 gsm to about 20 gsm, specifically including all values within these ranges and any ranges created thereby. The lower nonwoven layer 220 may have a Tensile Stiffness of from about 0.2 N/mm to about 2.0 N/mm, specifically including all values within these ranges and any ranges created thereby, as measured according to the CD Cyclic Elongation to 3% Strain Method. The lower nonwoven layer 220 may have a Strain to Break of greater than about 10%, or from about 10% to about 50%, or from about 20% to about 40%, specifically including all values within these ranges and any ranges created thereby, as measured according to the Strain to Break Method. The lower nonwoven layer may have a Permanent Strain of about 0.005 to about 0.018 mm/mm, specifically including all values within these ranges and any ranges created thereby, as measured according to the CD Cyclic Elongation to 3% Strain Method. The lower nonwoven layer may have a Thickness at 7 g/cm² pressure of from about 0.1 mm to about 1.3 mm, specifically including all values within these ranges and any ranges created thereby, as measured according to the Nonwoven Thickness—Pressure Method.

The fibers in the upper nonwoven layer 210 and the fibers of the lower nonwoven layer 220 may be different. The fibers of the upper nonwoven layer 210 and the fibers of the lower nonwoven layer 220 may be the same. The upper nonwoven layer may be a carded nonwoven. The upper nonwoven layer 210 may be air through bonded or hydroentangled. The upper nonwoven layer 210 may be free of spunbond material.

Suitable upper and/or lower nonwoven layer materials 210, 220 may bend and recover their original shape following the bending force. Flimsy or highly flexible materials readily bend at low peak force (load) and with low bending energy. Unsuitable materials, while readily bending, do not have sufficient recovery energy and so retain a deformed, bent state because of insufficient recovery energy. Suitable materials have sufficient energy to recover their initial pre-bent state. The materials with sufficient bending recovery energy may be considered resilient upper and lower nonwoven layers. Particularly suitable upper nonwoven layers may have a Dry Recovery Energy of greater than about 0.03 N*mm, or from about 0.03 N*mm to about 1 N*mm, or from about 0.04 N*mm to about 0.5 N*mm, specifically including all values within these ranges and any ranges created thereby. Particularly suitable upper nonwoven layers may have a Dry Bending Energy of less than about 1.6 N*mm, or less than about 1.1 N*mm, specifically including all values within these ranges and any ranges created thereby.

The upper and lower nonwoven layers 210, 220 may comprise natural fibers. Some examples of suitable natural fibers may include eucalyptus, cotton, flax, bamboo, wheat straw, red algae and/or other seaweeds, and hemp. Additionally, natural fibers include modified natural fibers of any suitable composition may be selected. Some examples of suitable modified natural fibers may include rayon, viscose, or combinations thereof. Suitable upper and lower nonwovens 210, 220 may comprise from about 60% to about 100%, or from about 70% to about 100% cellulose fibers such as cotton or regenerated cellulosic fibers, such as rayon and/or viscose, specifically including all values within these ranges and any ranges created thereby, synthetic fibers, or from about 0 to about 40%, or from about 0 to about 30%, specifically including all values within these ranges and any ranges created thereby.

The upper and lower nonwoven layers 210, 220 may comprise polymer fibers. Some examples of suitable polymer fibers may include bi-component fibers comprising polyethylene (PE) and polyethylene terephthalate (PET) components or polyethylene terephthalate and co-polyethylene terephthalate components. The components of the bi-component fiber may be arranged in a sheath-core configuration, a side-by-side configuration, an eccentric sheath-core configuration, a trilobal arrangement, or any other desired configuration. The polymer fibers may include bi-component fibers having PE/PET components arranged in a concentric, sheath-core configuration, wherein the polyethylene component forms the sheath. Suitable upper and lower nonwoven fibers may be selected from PET (polyethylene terephthalate), PP (polypropylene), a BiCo (Bicomponent fiber) selected from PE/PP (PE sheath and PP core) and/or PE/PET (PE sheath PET core), PLA (polylactic acid), cellulosic fibers, and combinations thereof. Polymer fibers may be included to help provide structural integrity to the upper and lower nonwovens. The polymer fibers may help increase structural integrity of the upper and lower nonwovens in both a machine direction (MD) and in a cross-machine direction (CD), which may facilitate web manipulation during processing of the upper and lower nonwovens for incorporation into a pad.

The polymer fibers may allow the nonwoven layers to hold their shape and resist plasticizing when wet, thereby making the nonwoven layers resilient. The upper nonwoven layer and the lower nonwoven layer may be attached to the inner core layer through the application of a core construction adhesive that is applied either directly to the inner core layer or to at least one of the upper nonwoven layer and the lower nonwoven layer, via a conventional spray or slot coating application. It is to be appreciated that the core construction adhesive allows the layers to be joined but does not adversely interfere with the flow of fluid to the inner core layer.

While other materials may be useful in creating a resilient structure, it is believed that the stiffness of a PET core component in a sheath-core fiber configuration may be useful for imparting resilience to the upper and lower nonwovens. In synergistic combination, a PE sheath component, having a lower melting temperature than the PET core component, may be utilized to provide inter-fiber melt/fusion bonding, effected via heat treatment of the precursor batt. This can help provide tensile strength to the web in both the MD and CD. Such inter-fiber bonds may serve to reduce fiber-to-fiber sliding, and thereby further contribute to imparting shape stability and resiliency to the material even when it is wetted. Where a relatively higher weight fraction of polymer fibers is included, more connections within the structure may be created via heat treatment. However, too many connection points may impart greater stiffness to the upper and lower nonwovens than may be desirable. For this reason, selecting the weight fraction of the polymer fibers may involve prioritizing and balancing competing needs for stiffness and softness in the upper and lower nonwovens.

The resilient fibers may help the upper and lower nonwovens 210, 220 maintain permeability and compression recovery. The upper and lower nonwovens 210, 220 may comprise resilient fibers having varying cross sections, e.g., round and hollow spiral, and/or may comprise resilient fibers having varying sizes. Suitable fibers may be resilient and may be spun from any suitable thermoplastic resin, such as polypropylene (PP), polyethylene terephthalate (PET), or other suitable thermoplastics known in the art. The average staple length of the resilient fibers may be selected to be in the range of greater than about 10 mm, from about 20 mm to about 100 mm, or about 30 mm to about 50 mm, or about 35 mm to about 50 mm, specifically including all values within these ranges and any ranges created thereby. The resilient fibers may have any suitable structure or shape. For example, the resilient fibers may be round or have other shapes, such as spiral, scalloped oval, trilobal, scalloped ribbon, and so forth. Further, the resilient fibers may be solid, hollow, or multi-hollow. The resilient fibers may be solid and round in shape. In other suitable examples, resilient polymer fibers may include polyester/co-extruded polyester fibers. Other suitable examples of resilient polymer fibers may include bi-component fibers such as polyethylene/polypropylene, polyethylene/polyethylene terephthalate, polypropylene/polyethylene terephthalate bicomponent fibers. These bi-component fibers may have a sheath/core configuration.

The resilient polymer fibers may be polyethylene terephthalate (PET) fibers, or other suitable non-cellulosic fibers known in the art. PET fibers may be imparted with any suitable structure or shape. For example, the PET fibers may be round or have other shapes, such as spiral, scalloped oval, trilobal, scalloped ribbon, hollow spiral, and so forth. The PET fibers may be solid, hollow or multi-hollow. In one particular example, PET fibers may be hollow in cross section and have a curl or spiral configuration along their lengths. Optionally, the resilient polymer fibers may be spiral-crimped or flat-crimped. The resilient polymer fibers may have an average crimp count of about 4 to about 12 crimps per inch (cpi), or about 4 to about 8 cpi, or about 5 to about 7 cpi, or about 9 to about 10 cpi specifically including all values within these ranges and any ranges created thereby. Particular non-limiting examples of resilient polymer fibers may be obtained from Wellman, Inc. (Ireland) under the trade designations H1311 and T5974. Other examples of suitable resilient polymer fibers are disclosed in U.S. Pat. No. 7,767,598.

The stiffening fibers and resilient fibers should be carefully selected. For example, while the constituent polymers forming the stiffening polymer fibers and the resilient polymer fibers may have similarities, resilient polymer fiber composition should be selected such that their constituents' melting temperature(s) is/are higher than that of the bondable components of the stiffening polymer fibers. Otherwise, during heat treatment, resilient polymer fibers could bond to stiffening polymer fibers and vice versa, and thereby an overly rigid structure. To avoid this risk where the stiffening polymer fibers include bicomponent fibers, e.g., core-sheath configuration fibers with a sheath component of relatively lower melting temperature at which fusion bonding will occur, the resilient polymer fibers may comprise the constituent chemistry of only the core, which may be a polymer having a relatively higher melting temperature.

Nonwoven performance can be impacted by a combination of the nonwoven fiber choice, fiber properties, and how the fibers are arranged or connected. Nonwoven selection can impact the absorbent article's ability to recover its shape following compression, bending and extension (stretching) forces present in-use with body motion. If the fibers are short (less than about 10 mm), the fibers are likely to irreversibly rearrange under extension and compressive forces. The rearranging (changing their orientation/state) of fibers in a fiber matrix dissipates the tensile (elongation) or compressive forces so that the energy used to affect the deformation is no longer available for recovery to the original shape. Longer fiber networks (typically greater than about 10 mm but less than about 100 mm) can absorb the tensile/compressive forces typical of bodily motions along the fiber length and across the structure. As a result, the absorbed forces are available to recover the structure to its original state. Longer fiber networks composed of finer fibers (typically less than about 15 microns to about 20 microns) more readily elongate and compress. As a result, the core structure can deform more readily (and to a higher degree) but the energy associated with these deformations is relatively small and insufficient to carry the structure back to its original state. Thicker fiber, such as greater than about 20 microns or about 2.0 DTex to about 10 DTex, are both flexible under bodily forces but provide sufficient fiber and web recovery energy to return the structure to its original state.

The fiber arrangement in a long fiber network from a structural standpoint can impact the performance of the absorbent articles containing these nonwovens. Long fiber webs of thicker fibers are typically loftier than a conventional thin spunbond nonwoven web composed of continuous fine fibers that are closely spaced and physically bonded together. Creating a web of thicker fibers arranged in a more randomized orientation such as those that can be achieved via carding, hydro-entangling, and needling are able to elongate and compress, whereby the fibers only temporary adjust their arrangement (space between the fibers exist for these arrangements) and are able to carry/store the deformation forces and this energy is available for recovering the structural shape. Additionally, finer (less than about 2.0 DTex) synthetic fibers such as BiCo and PP fibers commonly found in spunbond are closely spaced, relatively parallel aligned and closely bonded together. The bonded fibers within these spunbond webs are so interconnected (with closely spaced point bonds) that in tensile (elongation) the fibers at the polymer level are forced to stretch, resulting in polymer chains within the fiber permanently rearranging. As a result, the fibers themselves potentially remain permanently elongated (permanently strained) and are no longer able to recover to their initial state.

Suitable nonwoven material examples may include, but are not limited to, the following materials: (i) a 40 gsm carded resilient nonwoven material produced by Yanjan China (material code; ATB Z87G-40-90) which is a carded nonwoven composed of a blend of 60% 2 DTex and 40% 4 DTex BiCo (PE/PET) fibers. The fibers are bonded (ATB=Through ‘hot’ Air Bonded) to create a wet resilient network. Without being limited by theory, it is believed that because of the presence of the 4 DTex BiCo fibers and the fiber-to-fiber bonded BiCo network, the material has a low Permanent Strain (less than about 0.013 mm/mm) in the CD Cyclic Elongation to 3% Strain Method and a sufficient Dry Recovery Energy (greater than about 0.03 N*mm) in the Dry CD Ultra Sensitive 3 Point Bending Method; (ii) a 55 gsm resilient spunlace material produced by Sandler Germany (material code: 53FC041001), which is a hydro-entangled nonwoven that is produced via a carding step (like the nonwoven described above) followed by hydro-entangling with an elevated drying step (as described in U.S. Patent Publication No. 2020/0315873A1) that creates both an entangled and BiCo bonded resilient network. The material comprises a fiber blend of 30% 10 DTex HS-PET, 50% 2.2 DTex BiCo (PE/PET), and 20% 1.3 DTex rayon. The material has a low Permanent Strain (less than about 0.013 mm/mm) in the CD Cyclic Elongation to 3% Strain Method and a sufficient Dry Recovery Energy (greater than about 0.03 N*mm) in the Dry CD Ultra Sensitive 3 Point Bending Method; and (iii) a 50 gsm resilient spunlace material produced by Sandler Germany (material code: 53FC041005 opt82), which is a hydro-entangled nonwoven that is produced via a carding step (like the nonwoven described above) followed by hydro-entangling with an elevated drying step (as described in U.S. Patent Publication No. 2020/0315873A1) that creates both an entangled and BiCo bonded resilient network. The material comprises a fiber blend of 60% 5.8 DTex BiCo (PE/PET), 20% 3.3 DTex tri-lobal ‘structural’ rayon, and 20% 1.3 DTex rayon. The material has a low Permanent Strain (less than about 0.013 mm/mm) in the CD Cyclic Elongation to 3% Strain Method and a sufficient Dry Recovery Energy (greater than about 0.03 N*mm) in the Dry CD Ultra Sensitive 3 Point Bending Method. While this material has 40% rayon that can soften when wet, the use of structural tri-lobal rayon fibers helps structural stability in the wet state.

The inner core layer 200 described herein may be a unitary structure. As used herein, “unitary structure” means that the inner core layer 200 is continuous and is constructed from essentially one type of material, this being essentially the same material, or essentially the same combination of two or more materials throughout the inner core layer 200. Variations in density and concentration of the material may occur, but these are limited to those which may be obtained without incorporation of regions which have been formed separately and then physically joined together. For example, when the inner core layer 200 comprises a liquid absorbent material with multiple different fiber types e.g., cellulosic fibers and superabsorbent polymers, the relative concentrations of superabsorbent particles and cellulosic fibers may be different in different parts of the inner core layer 200. However, when the construction is unitary, the inner core layer 200 does not, for instance, include layers or laminates of a different composition. Likewise, variations in the density or concentration of various components across the longitudinal direction, the lateral direction, or the thickness direction of the inner core layer 200 may occur. Yet the inner core layer 200 should not include areas or layers of different composition which are formed separately and later joined together, or areas of the same or different material that are physically separated by areas of substantially no basis weight of liquid absorbent material.

The inner core layer 200 may be produced in an airlaying process. Streams of one or more of cellulose and superabsorbent polymer are carried on a fast moving airstream and deposited into a three dimensionally shaped pocket on a rotating forming drum with a vacuum below to draw the cellulose and/or superabsorbent polymer into the pocket in a laydown station. This shaped pocket provides the sculptural shape of the absorbent core structure. The upper nonwoven layer may be first introduced onto the forming drum and under the vacuum the upper nonwoven layer is drawn into the three-dimensional pocket shape on the drum. In this case, the cellulose and/or superabsorbent polymer material stream is deposited on the upper nonwoven layer directly in the forming station. Prior to entering the forming station, the nonwoven is coated with an adhesive to provide a stronger connection of the cellulose and/or superabsorbent polymer to the nonwoven layer. On exiting the laydown section, the lower nonwoven layer is combined with the upper nonwoven layer carrying the cellulose and/or superabsorbent polymer layer exiting the laydown section. This lower nonwoven is precoated with adhesive to enable a perimeter seal and to better integrate the cellulose and/or superabsorbent polymer without hindering the flow of liquid into the cellulose and/or superabsorbent polymer matrix. These adhesives are not represented in the Figures for simplicity.

The inner core layer 200 may comprise any of a wide variety of absorbent materials commonly used in absorbent articles. The absorbent material may include cellulosic fibers. For example, the inner core layer 200 may comprise comminuted wood pulp, which is generally referred to as airfelt. One suitable absorbent core material is an airfelt material which is available from Weyerhaeuser Company, Washington, USA, under Code No. FR516. Examples of other suitable absorbent materials for use in the absorbent core may include creped cellulose wadding; meltblown polymers including coform; chemically stiffened, modified or cross-linked cellulosic fibers; synthetic fibers such as crimped polyester fibers; peat moss; cotton, bamboo; absorbent polymer materials; or any equivalent material or combinations of materials, or mixtures of these.

The absorbent material may comprise a matrix comprising cellulosic fibers and absorbent polymer materials, such as superabsorbent particles, sometimes referred to herein as “fluff/AGM”. The absorbent material may be uniformly distributed. The absorbent material may be discontinuously distributed within the absorbent core structure 10, for example, as individual pockets or stripes of absorbent material separated from each other by material-free areas.

Absorbent polymer materials for use in absorbent articles typically comprise water-insoluble, water-swellable, hydrogel-forming crosslinked absorbent polymers which are capable of absorbing large quantities of liquids and of retaining such absorbed liquids under moderate pressure. The absorbent polymer material for the absorbent cores according to the present disclosure may comprise superabsorbent particles, also known as “superabsorbent materials” or as “absorbent gelling materials”. Absorbent polymer materials, typically in particle form, may be selected among polyacrylates and polyacrylate based materials, such as for example partially neutralized, crosslinked polyacrylates. The term “particles” refers to granules, fibers, flakes, spheres, powders, platelets and other shapes and forms known to persons skilled in the art of superabsorbent particles. In some aspects, the superabsorbent particles may be in the shape of fibers, i.e., elongated, acicular superabsorbent particles.

The inner core layer 200 may comprise at least one of cellulosic fibers and superabsorbent particles. The inner core layer may comprise from about 50% to about 85% cellulosic fibers, or from about 55% to about 80%, or from about 60% to about 75%, specifically including all values within these ranges and any ranges created thereby, all by weight of the inner core layer. The inner core layer 200 may comprise from about 10% to about 50% superabsorbent particles, or from about 15% to about 50%, or from about 20% to about 40%, or from about 25% to about 35% superabsorbent particles, specifically including all values within these ranges and any ranges created thereby, all by weight of the inner core layer. The inner core layer 200 may comprise from about 125 gsm to about 500 gsm cellulosic fibers, specifically including all values within these ranges and any ranges created thereby. The inner core layer 200 may comprise from about 125 gsm to about 300 gsm superabsorbent particles specifically including all values within these ranges and any ranges created thereby. The inner core layer 200 may comprise from about 50% to about 85% cellulosic fibers, specifically including all values within these ranges and any ranges created thereby, and from about 15% to about 50% superabsorbent particles, specifically including all values within these ranges and any ranges created thereby.

Referring still to FIG. 2B, upper nonwoven layer 210 may include a left side region 210a and a right side region 210b, and lower nonwoven layer 220 may include a left side region 220a and a right side region 220b. The inner core layer 200 may be contained within the upper nonwoven layer 210 and the lower nonwoven layer 220 by substantially sealing at least a left side region 210a, 220a and a right side region 210b, 220b of the upper nonwoven layer 210 and the lower nonwoven layer 220. The upper and lower nonwoven layers 210, 220 may extend outwardly from an inner core layer perimeter 200a and may be joined together with glue or other conventional bonding methods including, but not limited to, ultrasonic bonding, fusion bonding, crimping, and combinations thereof, to form a perimeter seal 230. The entire inner core layer 200 may be located inboard of the perimeter seal 230. The perimeter seal 230 may help to seal the absorbent material of the inner core layer 200 inside the upper and lower nonwoven layers 210, 220. The perimeter seal 230 may have a seal width WS, such as illustrated in FIG. 2B, of between about 1 mm and about 10 mm, or between about 2 mm and about 8 mm, or between about 3 mm and 6 mm. The seal width WS may be uniform or may vary about the perimeter of the inner core layer 200.

Referring to FIG. 3, the perimeter seal 230 may extend around the entire inner core layer perimeter 200a. Alternatively, the perimeter seal 230 may extend partially around the inner core layer perimeter 200a. The absorbent article 20 may include a front end seal 234 and a back end seal 235. The front end seal 234 and/or back end seal 235 may seal the topsheet, upper nonwoven layer, lower nonwoven layer, and the backsheet together. In some configurations, the front end seal 234 and/or the back end seal 235 may seal the topsheet and the backsheet. The front end seal 234 and/or the back end seal 235 may be a crimp seal.

The upper and lower nonwoven layers 210, 220 may be discrete materials that can be cut to approximately the size and shape of the inner core layer 200 so as to fit between the topsheet 110 and backsheet 130 but may not extend substantially into either the front end seal 234 or the back end seal 235. The upper and/or lower nonwoven layers 210, 220 may extend from the front edge of the absorbent article, through the front end seal 234 and the back end seal 235, to the back edge of the absorbent article.

The perimeter seal 230 may be positioned in at least the middle region 22 of the absorbent article 20 and/or the absorbent core structure 10. It is believed that the middle region 22 (located between the wearer's thighs during use) may be subjected to the most frequent and/or highest forces during use. It was found that the presence of at least a partial perimeter seal 230 at a left side region 210a and a right side region 210b of the upper nonwoven layer 210 and a left side region 220a and a right side region 220b of the lower nonwoven layer 220 external to the inner core layer 200 may help to ensure the upper and lower nonwovens 210, 220 maintain their structural function during physical deformations without separating, limiting any potential integrity and bunching issues.

As illustrated in FIGS. 3 and 4, the absorbent core structure 10 may have a non-rectangular perimeter. As illustrated in FIG. 3, the absorbent structure 10 may include substantially hourglass-shaped absorbent structure 10. In another example, referring now to FIG. 4, the absorbent core structure 10 may be shaped to define a tapering along its width towards the longitudinal axis 80. The absorbent core structure 10 may be shaped such as to conform to a wearer's inner thigh geometry, such as, for example, an hourglass shape, an offset hourglass shape (one end is wider than an opposite end and a narrowed mid-section between the ends), a bicycle seat shape (one end and central portion are narrower than the second end), an oval shape, or a trapezoid shape. It is to be appreciated that the absorbent core structure 10 may be any shape that allows the absorbent material to readily absorb and retain fluid, as previously discussed herein.

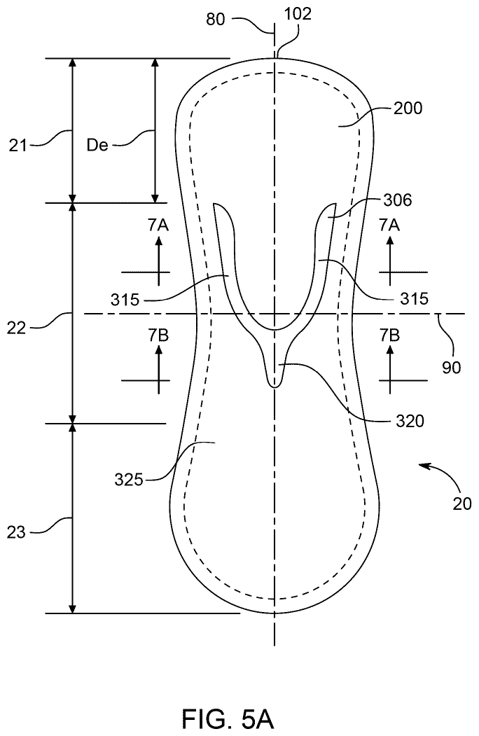

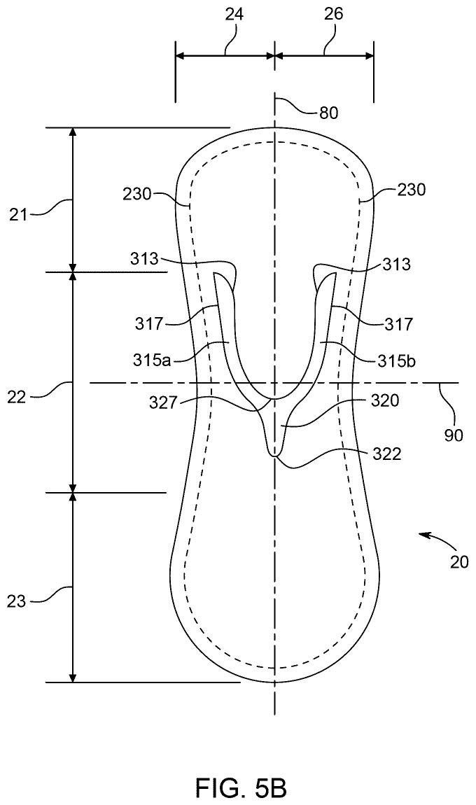

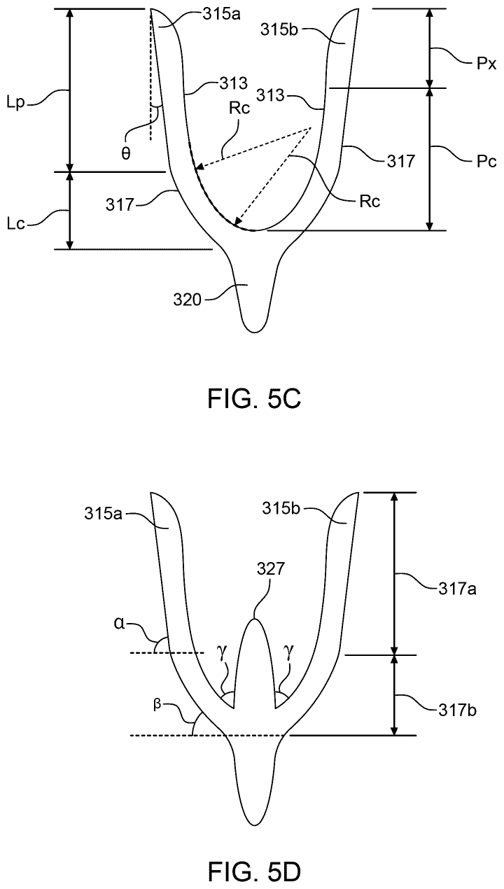

As illustrated in FIG. 5A, the absorbent article 20 described herein may further comprise a sculptural inner core layer 200 comprising a first absorbent zone 306 having a first basis weight and a second absorbent zone 325 having a second basis weight. As previously discussed herein, in the case of female genitalia, fluid first exits the body internally within the labial vestibule, in particular within the labial minora, prior to exiting through the labial majora. Fluid can exit the labial structure at the top, the bottom, and/or at the sides. Since the labial vestibule is curved, there typically exists a gap between the labia majora such that fluid exiting the labial minora is not captured by simply adding more absorbent material in the central region. Traditional “more in the middle” shapes rest atop of the labial structure and thus forms a bridge across the gap between the labial majora without actually capturing the fluid that resides and is moving from within the labial vestibule outwards. Additionally, typical bulky central regions may actually push the non-raised portions of the absorbent article away from the body, thus causing more gapping on the sides for fluid to spread and be felt by the consumer. The first absorbent zone 306 is shaped and configured to more effectively capture fluid as it exits the labial minora, as will be discussed in more detail herein. The first absorbent zone 306 and the second absorbent zone 325 may be formed by a three-dimensionally shaped distribution of absorbent material in the inner core layer 200. The first absorbent zone 306 may include one or more separate areas having substantially the same basis weight and/or substantially the same caliper. The first absorbent zone 306 may be substantially surrounded by the second absorbent zone 325. The first absorbent zone 306 may be at least partially surrounded by the second absorbent zone 325.

As illustrated in FIG. 5A, the first absorbent zone 306 may be a substantially wishbone shaped zone. More specifically, the first absorbent zone 306 may include two lateral side zones 315 and a central zone 320. The two lateral side zones 315 are positioned to at least partially surround the labia majora and capture the fluid from this area by maintaining contact with the wearer and sufficiently surrounding this area. The central zone 320 may be positioned between the two lateral side zones 315. The central zone 320 is positioned between the two lateral side zones 315 and extends into at least a portion of the perinium. It has been found that the movement of the wearer, such as from sitting to standing or laying down to being upright, causes a substantial risk of leakage because there is insufficient contact between the absorbent article and the perinium. Thus, the central zone 320 is shaped to continue the coverage of the first absorbent zone 306 about the labia majora and extend rearward into the perinium to in essence form a perinium dam and prevent fluid from leaking beyond this area of the wearer.

The two lateral side zones 315 may be symmetric with respect to a longitudinal axis 80 of the absorbent article 20 and asymmetric with respect to a lateral axis 90 of the absorbent article 20. The two lateral side zones 315 may extend in a direction substantially parallel to the longitudinal axis of the absorbent article 20. Similarly, the central zone 320 extends in a direction substantially parallel to the longitudinal axis of the absorbent article. The first absorbent zone 306 may be positioned substantially within the middle region 22 of the absorbent article. More specifically, the first absorbent zone 306 is positioned at a distance, De, measured parallel to the longitudinal axis 80 from the front edge 102 to a point on the lateral side zone 315 that is closest to the front edge 102. The distance, De, is from about 35 mm to about 75 mm, including all values within these ranges and any ranges created thereby. It has been found that positioning the first absorbent zone 306 at this distance, De, from the front edge 102 enables to the first absorbent zone 306 to be situated about the sides of the labial majora, which allows the absorbent article to readily absorb fluid resulting in reduced leakage. The width of each of the two lateral side zones 315 may vary along the longitudinal axis 80.

Referring to FIG. 5B, to sufficiently surround the labia majora, at least a portion of each of the two lateral side zones 315 may be curved. Further, each of the two lateral side zones 315 may include an outer edge 317 and an inner edge 313. The outer edge 317 may be positioned between the perimeter of the absorbent core structure 10 and the inner edge 313. The inner edge 313 may be positioned between the outer edge 317 and the longitudinal axis 80. Additionally, the two lateral side zones 315 may be symmetric about the longitudinal axis 80. A first lateral side zone 315a extends away from the longitudinal axis 80 towards the perimeter seal 230 of the absorbent structure 10 within a first region 24 of the absorbent structure and a second lateral side zone 315b extends away from the longitudinal axis towards the perimeter seal 230 on the opposite side of the absorbent structure within a second region 26 of the absorbent structure. Each of the first lateral side zone 315a and the second lateral side zone 315b do not intersect the longitudinal axis 80. The central zone 320 may be positioned between the first lateral side zone 315a and the second lateral size zone 315b. The central zone 320 may include a forward edge 327 and a rearward edge 322. The rearward edge 322 of the central zone 320 may be positioned inward from the outer edge 317 of each of the two lateral side zones 315. At least a portion of the central zone 320 may be continuous with at least a portion of the two lateral side zones 315. As used herein, “continuous” means that at least a portion of a first zone is connected to at least a portion of another zone so that there is no clear visual division or basis weight division between the connected portions of the zones. More specifically, the outer edge 317 of the first side zone 315a is continuous with a first portion of the reward edge 322 of the central zone 320 and the outer edge of the second side zone 315b is continuous with a second portion of the reward edge 322 of the central zone 320. Similarly, the inner edge 313 of the first side zone 315a is continuous with a first portion of the forward edge 327 of the central zone 320 and the inner edge of the second side zone 315b is continuous with a second portion of the forward edge 327 of the central zone 320. Without being bound by theory, it is believed that having at least a portion of the central zone 320 be continuous with at least a portion of the two lateral side zones 315 may provide improved leak protection when used with tightfitting undergarments.

Each of the first lateral side zone 315a and the second lateral side zone 315b may be shaped to substantially surround that labial majora. It has been found the each of the first lateral side zone 315a and the second lateral side zone 315b having various curved and angled portions allows for better contact with the wearer and fits a more diverse group of consumers. The inner edge 313 of each of the lateral side zones 315 may include a curvilinear portion. The curvilinear portion may comprise at least one of a convex portion relative to the longitudinal axis 80 and a concave portion relative to the longitudinal axis 80. For example, referring to FIG. 5C, the two lateral side zones 315 could have both a convex portion, Px, relative to the longitudinal axis 80 and a concave portion Pc relative to the longitudinal axis 80. As used herein “concave” means a shape formed by an arc that curves substantially inward relative to an axis, and “convex” means a shape formed by an arc that curves substantially outward relative to an axis. Each of the concave portion and the convex portion may have a radius of curvature. The concave portion may have a radius of curvature, Rc, of from about 35 mm to about 60 mm specifically including all values within these ranges and any ranges created thereby. It is to be appreciated that the inner edges 313 may each be linear and not curvilinear. For example, the inner edge may include a first linear portion at a first angle and a second linear portion at a second angle, wherein the first angle and the second angle are different.

The outer edges 317 of each of the lateral side zones 315 may be the same shape as or substantially the shape same as each of the inner edges 313 of the lateral side zones 315, or, as illustrated in FIG. 5C, the outer edges 317 of each of the lateral side zones 315 may be a different shape than the inner edges 313 of each of the lateral side zones 315. The outer edge 317 of the lateral side zones 315 may include a linear portion, Lp, and a curvilinear portion, Lc. The linear portion may form an angle, θ of from about 5 degrees to about 25 degrees, or from about 5 degrees to about 15 degrees measured from a line parallel to the longitudinal axis 80. The curvilinear portion, Lc, of the outer edge 317 may be a convex portion relative to the longitudinal axis. It is to be appreciated that alternatively the outer edge 317 may include a first curvilinear portion and a second curvilinear portion and no linear portion. Similarly, the outer edge 317 may include a curvilinear portion and no linear portion. Further, the outer edge 317 may include a first linear portion at a first angle and a second linear portion at a second angle, wherein the first angle and the second angle are different. Without being bound by theory, it is believed that having an angle, θ of from about 5 degrees to about 25 degrees, or from about 5 degrees to about 15 degrees, the lateral side zones 315 may be able to better to fit around the sides of the labial majora and better prevent fluid that exits the labial minora from leaking around the lateral sides of the absorbent article. It is to be appreciated that each of the edges of the first side zone and the second side zone may include the aforementioned features. Although FIG. 5C illustrates the portions of the edges with respect to the first side zone, this is also applicable to the second side zone.

As previously discussed, the inner edge 313 of each of the two lateral side zones 315 and a forward edge 327 of the central absorbent zone 320 may form a continuous curve. Central zone 320 may include a concave portion relative to the longitudinal axis 80 and/or a concave portion relative to the lateral axis 90. The outer edge 317 of each of the two lateral side zones 315 and the rearward edge 322 of the central zone 320 may form a continuous curve or may meet to form angles. The rearward edge 322 of the central zone 320 may be curvilinear and, more specifically, may be concave relative to each of the longitudinal axis 80 and/or the lateral axis 90.

Referring to FIG. 5D, the forward edge 327 of the central zone 320 may be curvilinear and, more specifically, may be concave relative to each of the longitudinal axis 80 and/or the lateral axis 90. The inner edge 313 of each of the two lateral side zones 315 and the forward edge 327 of the central zone 320 may form a continuous curve or may meet to form angles, γ. The angle γ may be greater than about 5 degrees to about 90 degrees or greater than about 15 degrees and less than about 75 degrees or less than about 90 degrees or less than about 75 degrees or less than about 45 degrees or less than about 30 degrees. The forward edge 327 of the central zone 320 may be curvilinear and, more specifically, may be concave relative to each of the longitudinal axis 80 and/or the lateral axis 90.

The outer edge 317 of each of the two lateral side portions may comprise a first portion 317a and a second portion 317b. The first portion 317a may be closer to the front edge 102 of the absorbent article 20 and the second portion 317b may be closer to the rear edge 103 of the absorbent article 20. The first portion 317a may have a first angle, a, with respect to a line parallel to the lateral axis 90 and the second portion 317b may have a second angle, β, with respect to a line parallel to the lateral axis 90. The first angle and the second angle may be the same. The first angle α and the second angle β may be different. The first angle may be from about 65 degrees to about 85 degrees and the second angle may be from about 30 degrees to about 60 degrees specifically including all values within these ranges and any ranges created thereby. Without being bound by theory, it is believed that having a first angle, a, from about 65 degrees to about 85 degrees and a second angle β, from about 30 degrees from about 60 degrees, the lateral side zones 315 may be able to better to fit around the sides of the labial majora and better prevent fluid that exits the labial minora from leaking around the lateral sides of the absorbent article.

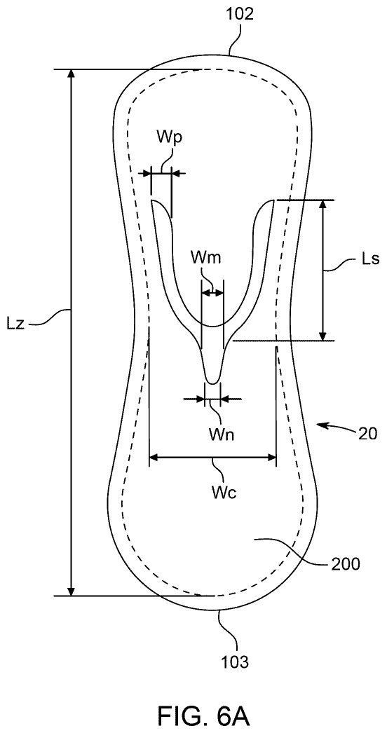

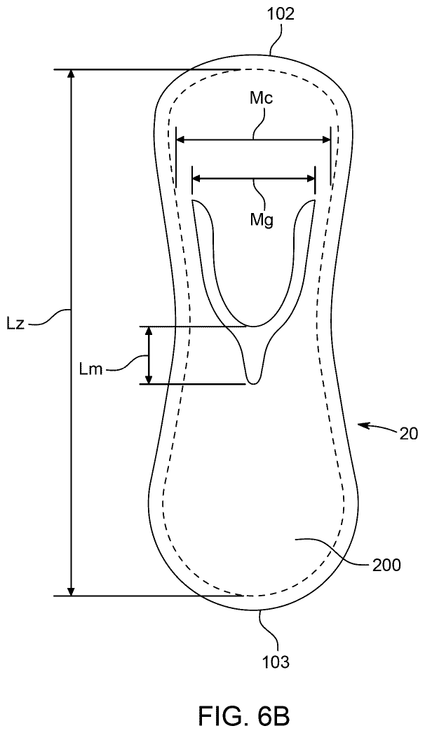

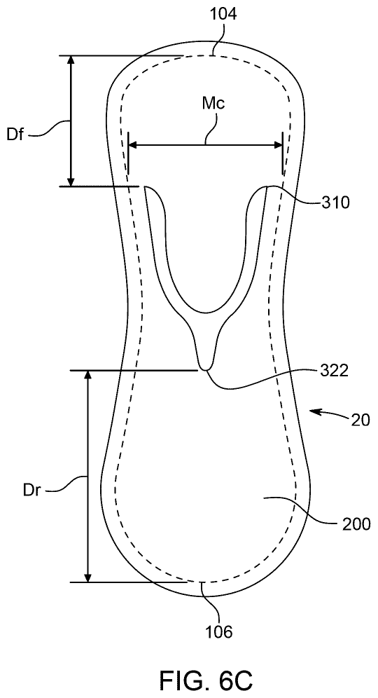

Referring to FIG. 6A, each of the two lateral side zones 315 may have a longitudinal length, Ls, measured along a line parallel to the longitudinal axis 80 from a point of the lateral side zones 315 closest to the front edge 102 to a point of the lateral side zones 315 closest to the rear edge 103, of from about 50 mm to about 100 mm, or from about 60 mm to about 90 mm, specifically including all values within these ranges and any ranges created thereby. Each of the two lateral side zones 315 may have a longitudinal length, Ls, of from about 10% to about 30%, or from about 10% to about 25%, or from about 12% to about 25%, specifically including all values within these ranges and any ranges created thereby, of the longitudinal length of the inner core layer 200, Lz, as measured along a line parallel to the longitudinal axis 80 from the outward most point of the front edge 102 to the outward most point of the rear edge 103. The longitudinal length of the inner core layer 200, Lz, may vary greatly as some users may desire absorbent articles having much greater longitudinal lengths than other users, and this may cause the relative length of the two lateral side zones 315, Ls, compared to the inner core layer length, Lz, to vary considerably. However, in some combinations, absorbent articles with greater inner core lengths may also have lateral side zones 315 with greater lengths.

Each of the two lateral side zones 315 have a lateral width measured parallel to the lateral axis 90. The lateral width of the two lateral side zones 315 may be uniform along the length of the two lateral side zones 315, or the lateral width may be variable along the length of the two lateral side zones 315. The lateral width of each of the two lateral side zones 315 at the widest point, Wp, may vary from about 4 mm to about 12 mm. The width of the two lateral side zones 315 may taper to form a point on the two lateral side zones 315, wherein the point formed is at the edge of the lateral side zones 315 that is closest to the front edge 102.

The width of the central absorbent zone 320 may vary along the longitudinal axis 80. The central absorbent zone 320 may taper so that the width of the central absorbent zone becomes narrower as the central absorbent zone 320 comes closer to the rear edge 103 of the absorbent article 20. Stated another way, a first portion of the rearward edge 322 of the central zone 320 converges toward a second portion of the rearward edge 322 such the first portion and the second portion meet along the longitudinal axis 80. Referring back to FIG. 5D, in embodiments where the inner edge 313 of each of the two lateral side zones 315 and the forward edge 327 of the central zone 320 form a continuous curve or meet to form angles, the forward edge 322 may also taper so that the width of the central absorbent zone becomes narrower as the central absorbent zone 320 comes closer to the front edge 102 of the absorbent article 20. The central absorbent zone 320 may have a width, Wm, of from about 9 mm to about 25 mm specifically including all values within these ranges and any ranges created thereby, when measured along a line parallel to the lateral axis 90 at the widest portion of the central absorbent zone 320. The central absorbent zone 320 may have a width, Wn, of from about 5 mm to about 15 mm, specifically including all values within these ranges and any ranges created thereby, when measured along a line parallel to the lateral axis 90 at a position different from the widest portion of the central absorbent zone 320. The width of the central absorbent zone 320, Wm, may cover from about 12% to about 30%, preferably from about 15% to about 25% of the inner core layer 200 width, Wc, measured along the same line used to measure Wm, specifically including all values within these ranges and any ranges created thereby, when measured parallel to the lateral axis 90.

Referring to FIG. 6B, a maximum gap, Mg, between the two lateral side zones 315 may be from about 40 mm to about 70 mm, or from about 45 mm to about 60 mm, specifically including all values within these ranges and any ranges created thereby, when measured along a line parallel to the lateral axis 90 at the widest point between the two lateral side zones 315. A maximum gap between the two lateral side zones 315 may be from 50% to 80% of a width of the inner core layer 200, Mc, measured along the same line used to measure Mg, specifically including all values within these ranges and any ranges created thereby, when measured parallel to the lateral axis 90.

The central absorbent zone 320 may have a longitudinal length, Lm, of from about 15 mm to about 50 mm, specifically including all values within these ranges and any ranges created thereby, wherein the longitudinal length, Lm, is measured along a line parallel to the longitudinal axis 80 at the longest length of the central absorbent zone 320. In embodiments where the inner edge 313 of each of the two lateral side zones 315 and the forward edge 327 of the central zone 320 form a continuous curve or meet to form angles, the forward edge 327 may protrude from about 10 mm to about 30 mm beyond the portion the inner edge 313 that is closest to the rear edge 103 so that interlabial penetration of the front edge 327 is minimized. The central absorbent zone 320 may have a longitudinal length, Lm, of from about 10% to about 25% of the longitudinal length of the inner core layer 200, Lz, specifically including all values within these ranges and any ranges created thereby, as measured along a line parallel to the longitudinal axis 80. However, as the longitudinal length of the inner core layer 200, Lz, may vary greatly as some users may desired absorbent articles having much greater longitudinal lengths than other users, the relative length of central absorbent zone 320, Lm, compared to the longitudinal length of the inner core layer 200, Lz, may vary considerably. The central absorbent zone 320 may have a length, Lm, that is from about 15% to about 50%, specifically including all values within these ranges and any ranges created thereby, of the length of the two lateral side zones 315, Ls.

Referring to FIG. 6C, the forward most portion(s) 310 of the two lateral side zones 315 (i.e. the portion of the two lateral side zones 315 closest to the front edge 104) may be positioned a distance, Df, of from about 25 mm to about 45 mm from front edge 104 of the inner core layer 200, when measured along a line parallel to the longitudinal axis 80, specifically including all values within these ranges and any ranges created thereby. Rearward edge 322 of the first absorbent zone 306 may be positioned a distance, Dr, when measured along a line parallel to the longitudinal axis 80, of from about 25 mm to about 85 mm from rear edge 106 of the inner core layer 200, specifically including all values within these ranges and any ranges created thereby.

The basis weight of the first absorbent zone 306 may be greater than the basis weight of the second absorbent zone 325, creating a raised sculptural structure on the absorbent article 20. The first absorbent zone 306 may be shaped to at least partially fit around the sides of the labial majora and to at least partially fit into the perineum. Without being bound by theory, it is believed that shaping the central absorbent zone as described herein may allow the central absorbent zone to at least partially fit comfortably in the perineum and to act as a dam to better prevent fluid from traveling along the perineum, thereby better preventing discomfort to the user and better preventing leaks in the rear of the absorbent article. The first absorbent zone 306 and the second absorbent zone 325 may be highly compressible without being bulky or dense and thus can fit closely to a wide range of body shapes without discomfort. To achieve a sculptural zone structure, the first basis weight of the first absorbent zone 306 may be greater than the second basis weight of the second absorbent zone 325. The first basis weight and the second basis weight may differ by about 20% to about 100%. The first basis weight may be from about 180 gsm to about 450 gsm, or from about 230 gsm to about 400 gsm specifically including all values within these ranges and any ranges created thereby, as measured according to the Inner Core Layer Basis Weight Method. The second basis weight may be from about 150 gsm to about 320 gsm, or from about 200 gsm to about 300 gsm specifically including all values within these ranges and any ranges created thereby, as measured according to the Inner Core Layer Basis Weight Method. It is to be appreciated that the increase in basis weight in the first absorbent zone 306 is due to additional absorbent material in the inner core layer 200 positioned in the first absorbent zone 306 relative to the second absorbent zone 325.

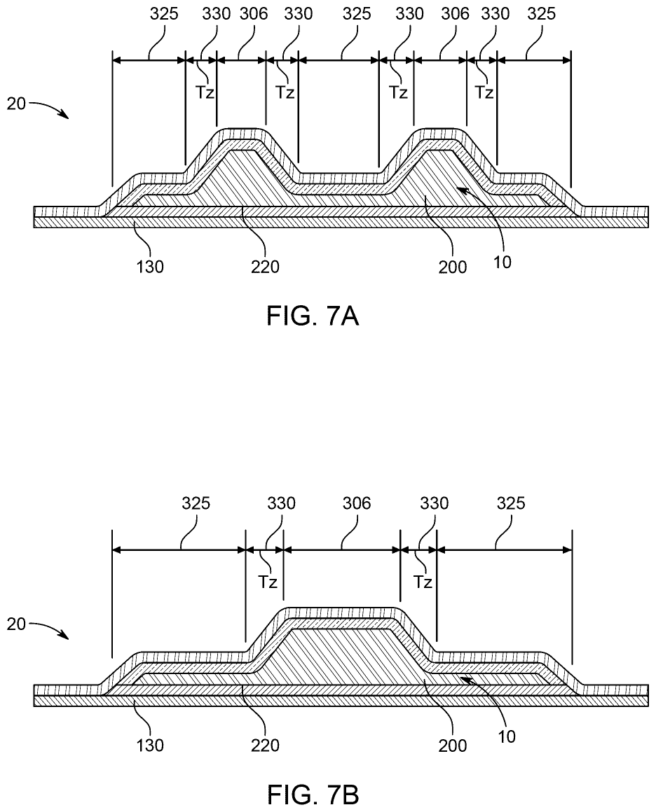

Referring to FIG. 7A and FIG. 7B, the first absorbent zone 306 may comprise a transition zone 330 that extends around the periphery of the first absorbent zone 306 where the basis weight of the inner core layer 200 gradually changes so that the basis weight of the inner core layer 200 may be greater in the first absorbent zone 306 than in the second absorbent zone 325. The upper nonwoven layer 210 is not in contact with the lower nonwoven layer 220 in the transition zone 330, as absorbent material of the inner core layer is disposed therebetween. The transition zone 330 may have a width (Tz) of from about 1 mm to about 5 mm, or from about 2 mm to about 3 mm, specifically including all values within these ranges and any ranges created thereby. The width of the transition zone may be measured from images obtained from microCT and analyzed by image analysis. Without being limited by theory, it is believed that a transition zone having the above described width may help to create the desired sculptural shape of the first absorbent zone 306 and allow it to fit closely to the wearer's body.

As previously discussed, the first absorbent zone 306 may be present in a middle region 22 of the absorbent article 20 and may be configured to extend about the labial vestibule of the wearer. It has been found that to accommodate the various wearers and be properly positioned, a total area of the first absorbent zone 306 may be from about 200 mm² to about 1080 mm², preferably from about 360 mm² to about 640 mm², specifically including all values within these ranges and any ranges created thereby.

In addition to the variation in area of the first absorbent zone 306 and the second absorbent zone 325, the first absorbent zone 306 may have a greater caliper than the second absorbent zone 325 to better maintain contact with the wearer. The absorbent article 20 may have varying caliper in the longitudinal and lateral directions created by the first absorbent zone 306 and the second absorbent zone 325. The absorbent article 20 may have a first caliper measured in the first absorbent zone 306, and a second caliper measured in the second absorbent zone 325. The first caliper may be from about 2 mm to about 6 mm, and the second caliper may be from about 1.0 mm to about 3.0 mm, specifically including all values within these ranges and any ranges created thereby, as measured according to the Absorbent Article Caliper, Basis Weight and Density Method. The first caliper of the first absorbent zone 306 may be greater than the second caliper of the second absorbent zone 325. The ratio of the first caliper to the second caliper may be from about 1.2 to about 2.5, specifically including all values within these ranges and any ranges created thereby.

As previously discussed, the ability of the absorbent article to maintain contact after the numerous compressions of the absorbent article due to the movement of the wearer is important for the absorbent article to continue to properly absorb fluid. To maintain contact, the absorbent article should exhibit a sufficient Energy of Z-Compression and bending modulus. The first absorbent zone 306 and/or the second absorbent zone 325 may exhibit an Energy of Z-Compression of from about 2.0 to about 8.0 N·mm, specifically including all values within these ranges and any ranges created thereby, as measured according to the Z-Compression Method. The Energy of Z-Compression of the first absorbent zone 306 may be substantially the same as the Energy of Z-Compression of the second absorbent zone 325. Without being limited by theory, it is believed that by having an Energy of Z-Compression of from about 2.0 to about 8.0 N·mm provides a high level of compressibility and conformability to gently envelope the user's genitalia and fit within the perinium.

The absorbent article may exhibit a MD Bending Modulus measured in the first absorbent zone 306 and the second absorbent zone 325 of from about 0.03 N/mm² to about 0.18 N/mm², or from about 0.03 to about 0.15 N/mm², specifically including all values within these ranges and any ranges created thereby as measured according to the Dry MD 3 Point Bend Method. The MD Bending Modulus of the first absorbent zone 306 may be substantially the same as the MD Bending Modulus of the second absorbent zone 325. Without being limited by theory, it is believed that by having a MD Bending Modulus of from about 0.03 N/mm² to about 0.18 N/mm² in the first absorbent zone 306 and the second absorbent zone 325 helps to provide both the integrity and the comfort of the first absorbent zone 306. If the MD Bending Modulus in the central absorbent zone 320 is too low, the material risks being too flimsy and rather than residing at least partially within the perinium it is likely to simply buckle over or bend away from the opening. If the MD modulus in the central absorbent zone is too high, the ability of the central absorbent zone 320 to comfortably fit and conform to the perinium can be compromised. If the MD Bending Modulus of the two lateral side zones 315 is too low, the material risks being too flimsy and may buckle or otherwise fail to at least partially fit around the sides of the labial majora. If the MD Bending Modulus is too high, the lateral zones may be uncomfortable about the sides of the labial majora and may be unable to conform to at least partially fit around the sides of the labial majora.

The average density of the absorbent article measured in the first absorbent zone 306 and the second absorbent zone 325 may be from about 0.045 g/cm³ and about 0.150 g/cm³ specifically including all values within these ranges and any ranges created thereby as measured according to the Absorbent Article Caliper, Basis Weight, and Density Method. The average density of the absorbent article measured in the first absorbent zone 306 and the second absorbent zone 325 may be substantially similar. The average density of the absorbent article measured in the second absorbent zone 325 may be within about 0% to about 20%, or about 0% to about 18%, or about 0% to about 10%, or about 0% to about 5%, specifically including all values within these ranges and any ranges created thereby, of the average density measured in the first absorbent zone 306.

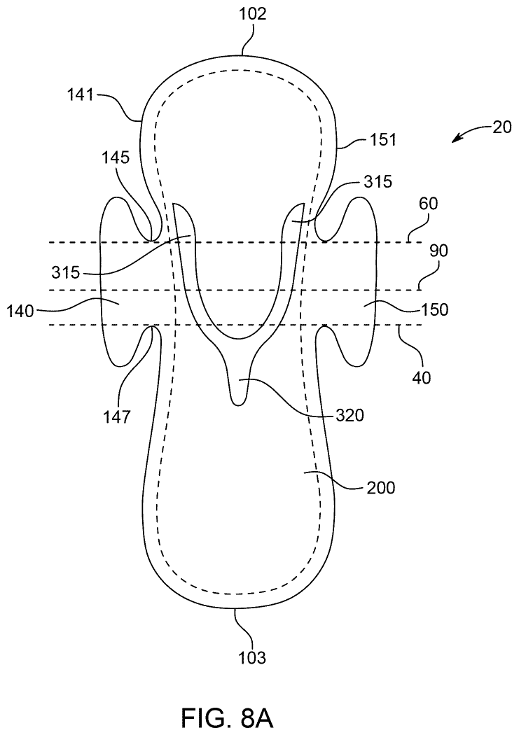

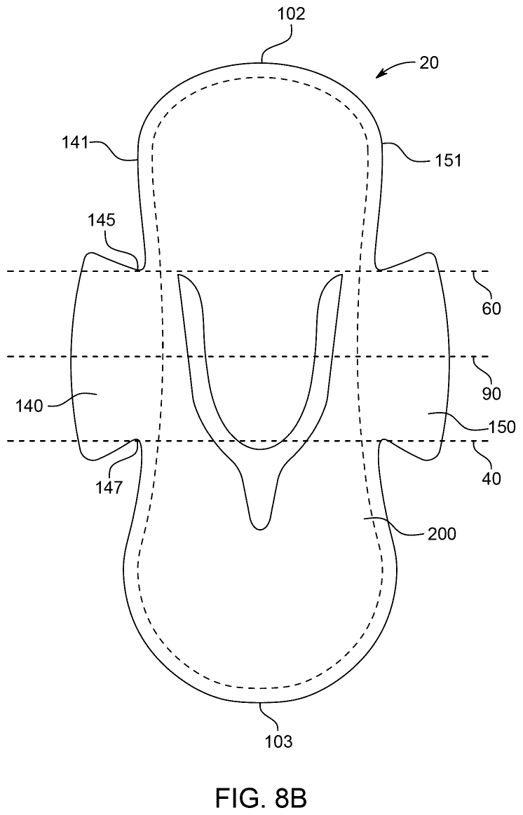

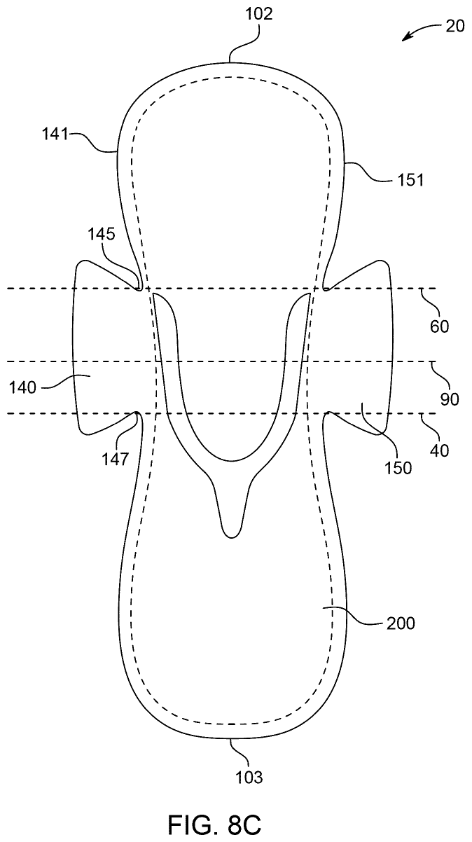

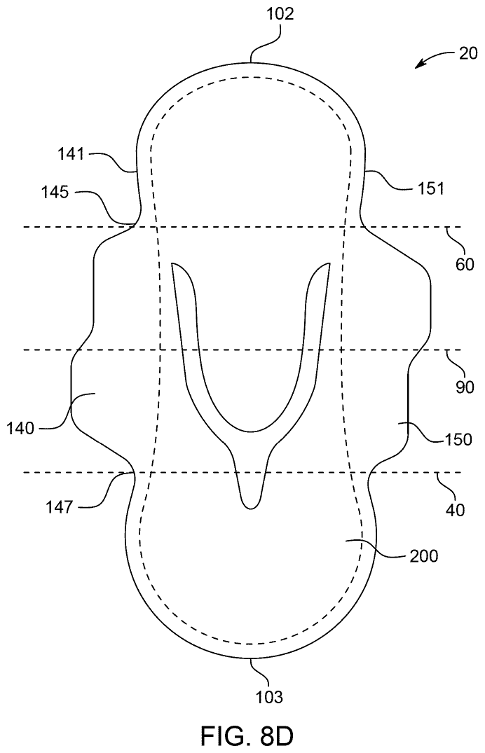

Referring to FIG. 8A-8D, the absorbent article 20 may include opposing wing portions 140, 150 on each side, extending laterally outward from a first longitudinal side 141 and a second longitudinal side 151 of the absorbent article. Wings may be symmetric or asymmetric such as disclosed in U.S. Pat. Nos. 11,083,647; 11,065,163; 11,246,770; and 11,058,591, which are herein incorporated by reference. Wings may include deposits of adhesive applied to their garment facing surfaces. The deposits of adhesive on the wings enable the user to wrap the wing portions through the leg openings of the undergarment and around the inside edges thereof, and adhere the wing portions to the outward-facing surface/underside of a user's undergarment in the crotch region, providing supplemental holding support for the absorbent article and helping guard the undergarment proximate the leg edges thereof against soiling.

At least a portion of the two lateral side zones 315 may be positioned forward (i.e. closer to the front edge 102 of the absorbent article 20) of the lateral axis 90, such as illustrated in FIG. 8A-8D. At least a portion of the two lateral side zones 315 may be positioned behind (i.e. closer to the rear edge 103 of the absorbent article 20) the lateral axis 90, such as illustrated in FIG. 8A-8D. A least a portion of the central absorbent zone may be positioned behind (i.e. closer to the rear edge 103 of the absorbent article 20) the lateral axis 90, such as illustrated in FIG. 8A-8D. Alternatively or additionally, at least a portion of the two lateral side zones 315 may be positioned forward of (i.e. closer to the front edge 102 of the absorbent article 20, as illustrated in FIG. 8A), or behind (i.e. closer to the rear edge 103 of the absorbent article, as illustrated in FIG. 8B-8D) a line 60 parallel to lateral axis 90 extending from a front extension point 145 of at least one of wings 140, 150. At least a portion of the two lateral side zones 315 may be positioned forward of (FIG. 8D), or behind (FIG. 8A-8C) a line 40 parallel to lateral axis 90 extending from a rear extension point 147 of at least one of wings 140, 150. At least a portion of the central absorbent zone 320 may be positioned forward of (FIG. 8D), or behind (FIG. 8A-8D) a line 40 parallel to lateral axis 90 extending from a rear extension point 147 of at least one of wings 140, 150. The first absorbent zone 306 may be positioned relative to the genitalia of the user so that leaks are minimized.

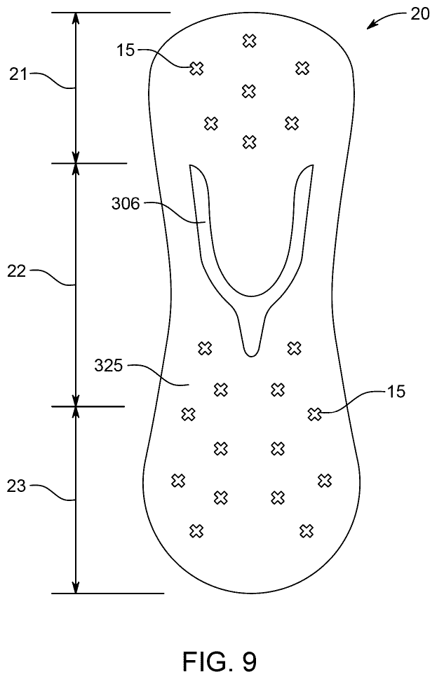

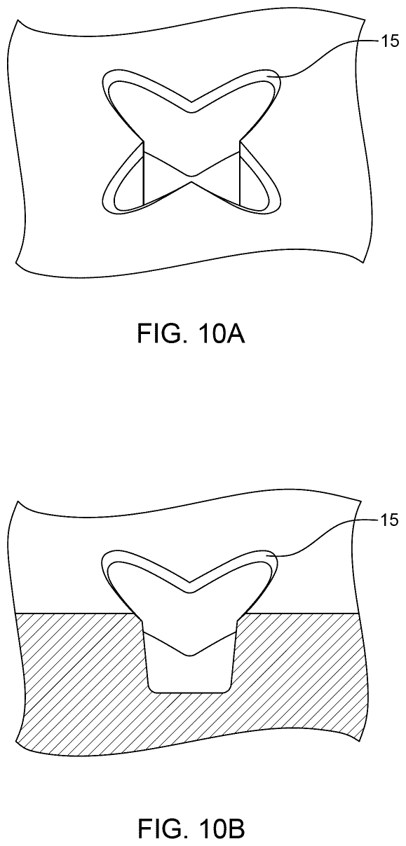

As illustrated in FIG. 9, the absorbent article 20 may further comprise a plurality of structural bond sites 15. FIGS. 10A and 10B show illustrations of example structural bond sites 15. FIG. 10A is a close up illustration of an example structural bond site 15. FIG. 10B is a cross-section view of the structural bond site 15 of FIG. 10A. The structural bond sites 15 may be symmetric and/or asymmetrical and may be any shape including, but not limited to, circles, ovals, hearts, diamonds, triangles, squares, stars, and/or X shapes. While the shape of the structural bond sites may be any shape, suitable shapes may be more detailed shapes such as asymmetrical shapes (versus simple dots). The structural bond sites 15 may be on the absorbent article and/or on the absorbent core structure. The structural bond sites may have a bond area of from about 2 mm² to about 5 mm², specifically including all values within these ranges and any ranges created thereby. The total structural bond area may be from about 0.5% to about 5%, or from about 0.75% to about 4.5%, or from about 1% to about 4% specifically including all values within these ranges and any ranges created thereby of the absorbent core structure, as measured according to the Structural Bond Sites Pattern Spacing and Area Measurement Method. The total structural bond area may be from about 1% to about 4% specifically including all values within these ranges and any ranges created thereby of absorbent article as measured according to the Structural Bond Sites Pattern Spacing and Area Measurement Method. The average distance between the structural bond sites may be from about 10 mm to about 32 mm specifically including all values within these ranges and any ranges created thereby. The average distance between the structural bond sites may be greater than about 20 mm. The structural bond sites may have a maximum width of from about 1 mm to about 6 mm, or from about 1.5 mm to about 5 mm, or from about 2 mm to about 4 mm specifically including all values within these ranges and any ranges created thereby. Without being limited by theory, it is believed that the average distance between structural bond sites and/or the size of the structural bond sites may help to maintain the structural integrity of the absorbent core structure without creating an undesirable stiffness that may inhibit the ability of the absorbent article to conform to the body.

The structural bond sites 15 may be distributed across the absorbent article and/or absorbent core structure or they may be clustered in regions of the absorbent article and/or absorbent core structure. The structural bond sites may be clustered in the middle region 22 of the absorbent article 20 and/or absorbent core structure 10. The middle region 22 of the absorbent article 20 and/or absorbent core structure 10 may be substantially free from structural bond sites 15 and may be surrounded by an area of structural bond sites and/or embossing. As shown in FIG. 9, the second absorbent zone 325 may comprise a plurality of structural bond sites 15 and the first absorbent zone 306 may be substantially free from structural bond sites 15.

The structural bond sites 15 may join the topsheet 110 to one or more layers of the absorbent article 20. For example, the structural bond sites 15 may join the topsheet 110 to the upper nonwoven layer 210, the inner core layer 200, and/or the lower nonwoven layer 220. Absorbent articles 20 and/or absorbent core structures 10 may comprise an upper nonwoven layer 210 and a lower nonwoven layer 220 that are closer together in the Z-direction at the structural bond sites 15 but are not melted together. Since these structural bond sites are not melted together, they may not be permanent in nature and rather may intermingle the materials within the structural bond site. The structural bond sites 15 may be substantially free of fusion bonds.

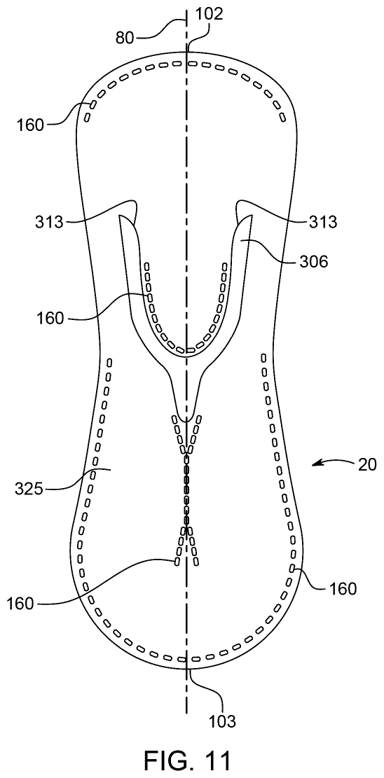

As shown in FIG. 11, the absorbent article 20 may also comprise one or more flex bond channel regions 160. As used herein, “flex bond channel region” refers to a generally elongated depression formed in at least a portion of an absorbent article, partially or entirely extending through the z-direction thickness of the absorbent article. Flex bond channel regions can reduce the thickness of the absorbent article in the z-direction and can act as preferential bending lines in the absorbent article, allowing the article to bend in particular directions so as to fit more closely to the wearer's body. Flex bond channel regions may also act as fluid wicking or fluid transport barriers that can reduce the potential for fluid to migrate to the absorbent article perimeter and cause a leak.

At least one flex bond channel region 160 may be positioned between a portion the first absorbent zone 306 and the second absorbent zone 325. In configurations where an inner edge of the two lateral side zones 315 and a front edge of the central zone 320 form a continuous curve, the flex bond channel region 160 may be positioned to follow the continuous curve of the first absorbent zone 306, and may be positioned from about 1 mm to about 10 mm away from the continuous curve, specifically including all values within these ranges and any ranges created thereby. The flex bond channel region 160 may be positioned in the transition zone 330. The flex bond channel region 160 may be positioned adjacent to the transition zone 330 in the second absorbent zone 325. The flex bond channel region 160 may be positioned about 1 mm to about 10 mm, or from about 3 mm to about 6 mm, outboard of the outermost edge of the transition zone 330. The flex bond channel region 160 may be positioned in second absorbent zone 325 so that at least a portion of the flex bond channel region 160 is on or parallel to longitudinal axis 80. The flex bond channel region 160 may be positioned adjacent the front edge 102. The flex bond channel region 160 may be positioned adjacent the rear edge 103.

The flex bond channel regions 160 may be a continuous depression and/or a series of individually compressed, closely spaced embossments. The flex bond channel region has a dry channel depth of from about 1.0 mm to about 4.5 mm and a channel width of from about 1.0 mm to about 3.0 mm, specifically including all values within these ranges and any ranges created thereby. The flex bond channels may include those described in patent publication WO2023/229888A1, the entirety of which is hereby incorporated by reference.

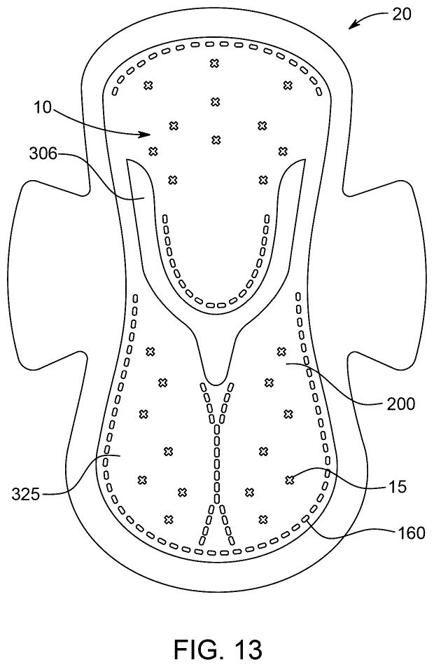

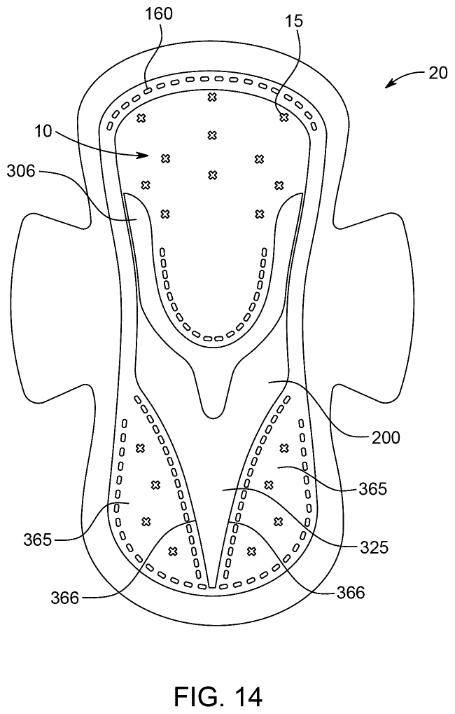

Based on the above disclosure, in some embodiments, an absorbent article 20 may have a first absorbent zone 306 and a second absorbent zone 325, as shown in FIG. 13. The absorbent article 20 may have a topsheet, a backsheet, and an absorbent core structure 10 with an upper nonwoven, a lower nonwoven, and an inner core layer 200. The total longitudinal length of the absorbent article at the longest portion may be 240 mm, and the lateral width of the absorbent article at the widest portion may be 86 mm, as determined according to the Dimensional and Geometric Measurement Method described herein. The topsheet may be a 24 gsm carded topsheet nonwoven. The upper nonwoven may be a 40 gsm carded hiloft nonwoven. The lower nonwoven may be a 17 gsm polypropylene spunbond nonwoven. The absorbent structure 10 may have a longitudinal length of 222 mm, a width at the widest portion of 80 mm, and a width at the narrowest point of 62 mm, as determined according to the Dimensional and Geometric Measurement Method described herein. The inner core layer 200 may be a blend of cellulose and AGM having a basis weight of 250 gsm cellulose and 50 gsm AGM for the top 3.8 mm of the first absorbent zone 306 and a blend of cellulose and AGM having a basis weight of 150 gsm cellulose and 60 gsm AGM for the bottom 2.7 mm of the first absorbent zone. The inner core layer 200 may be a blend of cellulose and AGM having a basis weight of 150 gsm cellulose and 60 gsm AGM for the second absorbent zone 325. The inner core layer 200 may have a caliper of 6.5 mm in the first absorbent zone 306 and a caliper of 2.7 mm in the second absorbent zone 325, as determined according to the Absorbent Article Caliper, Basis Weight, and Density Method described herein. The inner core layer 200 may have a density of 0.079 g/cm³ in the first absorbent zone 306 and a density of 0.077 g/cm³ in the second absorbent zone 325, as determined according to the Absorbent Article Caliper, Basis Weight, and Density Method described herein. The inner core layer 200 may have a longitudinal length of 210 mm, a width at the widest portions of 74 mm, and a width at the narrowest portion of 56 mm, as determined according to the Dimensional and Geometric Measurement Method described herein. De may be 30 mm, Lp may be 55 mm, Px may be 20 mm, Pc may be 50 mm, Mc may be 68 mm, Ls may be 75 mm, Wm may be 15 mm, Lm may be 30 mm, Mg may be 60 mm, and Dr may be 97 mm, as determined according to the Dimensional and Geometric Measurement Method described herein.

In some embodiments, an absorbent article 20 may have a first absorbent zone 306 and a second absorbent zone 325, as shown in FIG. 13. The absorbent article 20 may have a topsheet, a backsheet, and an absorbent core structure 10 with an upper nonwoven, a lower nonwoven, and an inner core layer 200. The total longitudinal length of the absorbent article at the longest portion may be 240 mm, and the lateral width of the absorbent article at the widest portion may be 86 mm, as determined according to the Dimensional and Geometric Measurement Method described herein. The topsheet may be a 24 gsm carded topsheet nonwoven. The upper nonwoven may be a 40 gsm carded hiloft nonwoven. The lower nonwoven may be a 17 gsm polypropylene spunbond nonwoven. The absorbent structure 10 may have a longitudinal length of 222 mm, a width at the widest portion of 80 mm, and a width at the narrowest point of 62 mm, as determined according to the Dimensional and Geometric Measurement Method described herein. The inner core layer 200 may be a blend of cellulose and AGM having a basis weight of 250 gsm cellulose and 50 gsm AGM for the top 3.8 mm of the first absorbent zone 306 and a blend of cellulose and AGM having a basis weight of 150 gsm cellulose and 60 gsm AGM for the bottom 2.7 mm of the first absorbent zone. The inner core layer 200 may be a blend of cellulose and AGM having a basis weight of 150 gsm cellulose and 60 gsm AGM for the second absorbent zone 325. The inner core layer 200 may be a blend of cellulose and AGM having a basis weight of 75 gsm cellulose and 25 gsm AGM for the third absorbent zone 365. The inner core layer 200 may have a caliper of 6.5 mm in the first absorbent zone 306, a caliper of 2.7 mm in the second absorbent zone 325, and a caliper of 1.5 mm in the third absorbent zone 365, as determined according to the Absorbent Article Caliper, Basis Weight, and Density Method described herein. The inner core layer 200 may have a density of 0.079 g/cm³ in the first absorbent zone 306, a density of 0.077 g/cm³ in the second absorbent zone 325, and a density of 0.066 g/cm³ in the third absorbent zone 365, as determined according to the Absorbent Article Caliper, Basis Weight, and Density Method described herein. The inner core layer 200 may have a longitudinal length of 210 mm, a width at the widest points of 74 mm, and a width at the narrowest point of 56 mm, as determined according to the Dimensional and Geometric Measurement Method described herein. De may be 30 mm, Lp may be 55 mm, Px may be 20 mm, Pc may be 50 mm, Mc may be 68 mm, Ls may be 75 mm, Wm may be 15 mm, Lm may be 30 mm, Mg may be 60 mm, and Dr may be 97 mm, as determined according to the Dimensional and Geometric Measurement Method described herein.

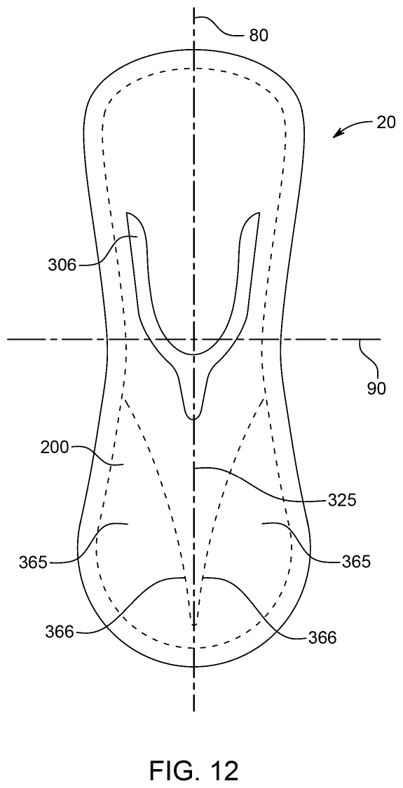

The absorbent article 20 may optionally comprise three or more absorbent zones. The absorbent article 20 may comprise a first absorbent zone 306, a second absorbent zone 325, and third absorbent zone 365, as shown in FIG. 12. The third absorbent zone may have a lower caliper and basis weight than the first absorbent zone 306 and/or the second absorbent zone 325. The third absorbent zone 365 may have a basis weight of from about 100 gsm to about 220 gsm. The third absorbent zone 365 may have a caliper of from about 1 mm to about 4 mm. The third absorbent zone 365 may provide increased flexibility to the absorbent article 365, and may allow the absorbent article 20 to bend so that the absorbent article can fit closely to the user. The third absorbent zone 365 may surround at least a portion of the second absorbent zone 325. The third absorbent zone 365 may be symmetric or asymmetric with respect to the longitudinal axis 80. Inner edges 366 of the third absorbent zone 365 may form a “v” shape, wherein the sides of the “v” may be linear or curvilinear. Inner edges 366 of the third absorbent zone 365 may form a “v” shape, wherein the sides of the “v” may be concave or convex with respect to the longitudinal axis 80.

A secondary topsheet (STS) layer, not shown in the figures, may optionally be included between the topsheet 110 and the absorbent core structure 10 to enable the absorbent core structure 10 to readily receive a sudden discharge of fluid, and after receipt, to wick it along x- and y-axis directions to distribute it across the underlying absorbent core structure 10. If included, an STS may be a nonwoven fibrous structure which may include cellulosic fibers, non-cellulosic fibers (e.g., fibers spun from polymer resin(s)), or a blend thereof. To accommodate the folding and lateral gathering of the absorbent article 20, and of the absorbent core structure 10, as described herein, the STS may be formed of a material that is relatively pliable (i.e., has relatively low bending stiffness).

A number of particular examples of suitable STS compositions and structures, as well as combinations thereof with suitable topsheet compositions and structures, are further described in U.S. application Ser. Nos. 16/831,862; 16/831,854; 16/832,270; 16/831,865; 16/831,868; 16/831,870; and Ser. No. 16/831,879; and U.S. Provisional Apps. Ser. Nos. 63/086,610 and 63/086,701. Additional suitable examples are described in U.S. Pat. No. 9,504,613; WO 2012/040315; and US 2019/0021917.

The absorbent article 20 may comprise an undergarment fastening component, such as an undergarment fastening adhesive, also referred to as a panty fastening adhesive, or components of a hook and-loop fastening system (such as VELCRO®). The absorbent article 20 may be provided with a undergarment fastening adhesive disposed on the garment-facing side of the backsheet 130 in order to provide a mechanism for the user to adhere the absorbent article to the inside of the undergarment in the crotch region thereof. The undergarment fastening adhesive may comprise any adhesive or glue used in the art for such purposes. These adhesives typically are pressure sensitive and remain tacky well below their application temperature. The undergarment fastening adhesive may be a pressure sensitive hot melt adhesive. When the absorbent article 20 is packaged for shipping, handling and storage prior to use, the undergarment fastening adhesive may be covered by one or more sheets of release film or paper that covers/shields the adhesive deposits from contact with other surfaces until the user is ready to remove the release film or paper and place the absorbent article in the undergarment for wear/use. The release film or paper may also function as an individualized packaging for the article or provide a disposal function as known in the art. Any commercially available release paper or film may be used. Suitable examples include BL 30 MG-A SILOX EIO, BL 30 MG-A SILOX 4 P/O available from Akrosil Corporation, and M&W films available from Gronau in Germany, under the code X-5432. The absorbent article may be packaged in a bi-folded or tri-folded state.

### Test Methods

### Layers of Interest

For any of the methods below in which all the component layers of an article will not be tested, the layers of interest may be separated using cryo-spray as needed from layers which will not be tested.

### Strain to Break Method

The force versus displacement behavior of a test specimen is measured on a universal constant rate of extension test frame (a suitable instrument is the MTS Alliance using TestSuite Software, as available from MTS Systems Corp., Eden Prairie, MN, or equivalent) equipped with a load cell for which the forces measured are within 1% to 99% of the limit of the cell. The sample is subjected to tensile elongation at a constant rate (mm/sec) until it breaks, and the percent strain to break is measured. All testing is performed in a room controlled at 23° C.±3 C.° and 50%±2% relative humidity and test samples are conditioned in this environment for at least 2 hours prior to testing.

The fixtures used to grip the test specimen are lightweight (<80 grams), vise action clamps with half cylinder steel versus rubber coated steel grip faces that are at least 40 mm wide. The fixtures are installed on the universal test frame and mounted such that they are horizontally and vertically aligned with one another.

Measurements are made on test specimens taken from rolls or sheets of the raw material, or test specimens obtained from a material layer removed from an absorbent article. When excising the material layer from an absorbent article, use care to not impart any contamination or distortion to the layer during the process. The excised layer should be free from residual adhesive and any fibers that may have transferred from underlying layers. To ensure that all adhesive and any transferred fibers are removed, soak the layer in a suitable solvent that will dissolve the adhesive and release any transferred fibers present without adversely affecting the material itself. One such solvent is THF (tetrahydrofuran, CAS 109-99-9, for general use, available from any convenient source). After the solvent soak, the material layer is allowed to thoroughly air dry in such a way that prevents undue stretching or other deformation of the material. After the material has dried, a test specimen is prepared as follows. The test specimen is cut from an area on the test material that is free of any folds or wrinkles. The test specimen is 100 mm long (parallel to the lateral axis, or intended lateral axis of the article) and 25.4 mm wide (parallel to the longitudinal axis, or intended longitudinal axis of the article). In like fashion, five replicate test specimens are prepared.

Prepare the universal test frame as follows. Set the initial grip to grip separation distance to a nominal gage length of 80 mm, then zero the crosshead. Program the test frame to move the grips closer together by an intentional slack of 1 mm to ensure no pretension force exists on the test specimen at the onset of the test. (During this motion, the specimen will become slack between the grips.) Next, the grips will move apart at a slack speed of 1 mm/s until the slack preload of 0.05 N is exceeded. (At this point, the crosshead position signal is used to compute the sample slack, the adjusted gage length, and the strain is defined at zero, 0.0). The grips will then move apart at a speed of 1 mm/s until the sample breaks or the extension limit of the instrument is exceeded.

The test is executed by inserting the test specimen into the grips such that the long axis of the specimen is parallel and centered with the motion of the crosshead. Start the test and continuously collect force (“load”) and displacement data at a data acquisition rate of 100 Hz.

Construct a graph of load (N) versus displacement (mm). Determine the peak load from the curve, then determine the break sensitivity as follows. Determine the crosshead position at which the load signal decreases by 75% after the peak load is reached, and record as specimen final length (Lf) to the nearest 0.01 mm. The initial length of the specimen is defined by the crosshead position when the slack preload of 0.05 N is exceeded, and this value is recorded as specimen initial length (Li) to the nearest 0.01 mm. Calculate the percent strain to break as follows, and record to the nearest 1 percent.

$$\% Strain to Break = ( ( Lf - Li ) / Li ) * 100$$

In like fashion, the procedure is repeated for all five replicate test specimens. The arithmetic mean of % strain to break among the five replicate test specimens is calculated and reported as % Strain to Break to the nearest 1 percent.

### Dry MD 3 Point Bend Method

The bending properties of an absorbent article test sample are measured on a universal constant rate of extension test frame (a suitable instrument is the MTS Alliance using TestSuite Software, as available from MTS Systems Corp., Eden Prairie, MN, or equivalent) equipped with a load cell for which the forces measured are within 1% to 99% of the limit of the cell. The test is executed on dry test specimens. The intention of this method is to mimic deformation created in the x-y plane by a wearer of an absorbent article during normal use. All testing is performed in a room controlled at 23° C.±3° C. and 50%±2% relative humidity.

The bottom stationary fixture consists of two cylindrical bars 3.175 mm in diameter by 110 mm in length, made of polished stainless steel each mounted on each end with frictionless roller bearings. These 2 bars are mounted horizontally, aligned front to back and parallel to each other, with top radii of the bars vertically aligned and are free to rotate around the diameter of the cylinder by the frictionless bearings. Furthermore, the fixture allows for the two bars to be moved horizontally away from each other on a track so that a gap can be set between them while maintaining their orientation. The top fixture consists of a third cylinder bar also 3.175 mm in diameter by 110 mm in length, made of polished stainless steel mounted on each end with frictionless roller bearings. When in place, the bar of the top fixture is parallel to and aligned front to back with the bars of the bottom fixture and is centered between the bars of the bottom fixture. Both fixtures include an integral adapter appropriate to fit the respective position on the universal test frame and lock into position such that the bars are orthogonal to the motion of the crossbeam of the test frame.

Set the gap (“Span”) between the bars of the lower fixture to 25 mm±0.5 mm (center of bar to center of bar) with the upper bar centered at the midpoint between the lower bars. Set the gage (bottom of top bar to top of lower bars) to 1.0 cm.

The thickness (“caliper”) of the test specimen is measured using a manually-operated micrometer equipped with a pressure foot capable of exerting a steady pressure of 0.1 psi±0.01 psi. The manually-operated micrometer is a dead-weight type instrument with readings accurate to 0.01 mm. A suitable instrument is Mitutoyo Series 543 ID-C Digimatic, available from VWR International, or equivalent. The pressure foot is a flat circular moveable face with a diameter no greater than 25.4 mm. The test specimen is supported by a horizontal flat reference platform that is larger than and parallel to the surface of the pressure foot. Zero the micrometer against the horizontal flat reference platform. Place the test specimen onto the platform, centered beneath the pressure foot. The pressure foot is lowered by hand with a descent rate of 3±1 mm/s until the full weight of the pressure is exerted onto the specimen. After 5 seconds elapse, the thickness is recorded as caliper to the nearest 0.01 mm.

The absorbent article samples are conditioned at 23° C.±3° C. and 50%±2% relative humidity two hours prior to testing. Remove the test sample from its outer wrapper, then remove the protective cover/release paper from the undergarment fastening adhesive on the garment facing side of the sample. Lightly apply talc powder to the adhesive to mitigate any tackiness. For each test sample, dry test specimens are prepared from two separate test locations on the sample from areas that are free from any folds or wrinkles, as follows. Referring to FIG. 5A, the first test specimen is obtained from a location on the test sample within the middle region 22 that comprises mostly the first absorbent zone 306 such that the center of the test specimen is the intersection of the lateral midpoint of the central zone 320 of the first absorbent zone and the longitudinal midpoint of the central zone 320 of the first absorbent zone 306. The second test specimen is obtained from the rear-most position possible (inbound of the perimeter seal 230) on the test sample within the rear region 23 that includes the second absorbent zone 325 such that the long side of the test specimen is parallel to the longitudinal axis 80 of the absorbent article. The second test specimen does not include any portion of any flex bond channel region or perimeter seal. The dry specimens are prepared for MD (machine direction) bending by cutting them to a width of 50.8 mm along the CD (cross direction; parallel to the lateral axis of the sample) and a length of 50.8 mm along the MD (parallel to the longitudinal axis of the sample), maintaining their orientation after they are cut, marking the body-facing surface (or the surface intended to face the body of a finished article), and labeling as “first absorbent zone” or “second absorbent zone” to correspond to each of the test locations. In the case where one of the test locations to be measured is not large enough to prepare a 50.8 mm by 50.8 mm test specimen per the test location requirements previously specified, then a test specimen of smaller dimensions may be used, adjusting the span such that it is about half of the specimen length. The dimensions of the test specimen prepared for the first absorbent zone must have the same dimensions as the test specimen prepared or the second absorbent zone, and the same span is used for both test locations. Measure the thickness of each of the test specimens, as described herein, and record as dry specimen caliper to the nearest 0.01 mm, denoting first or second absorbent zone for each. In like fashion, five replicate dry test specimens are prepared from the first and second absorbent zone test locations on five separate test samples.

Program the universal test frame for a flexural bend test, to move the crosshead such that the top fixture moves down with respect to the lower fixture at a rate of 1.0 mm/sec until the upper bar touches the top surface of the specimen with a nominal force of 0.02 N, then continue for an additional 12 mm. The crosshead is then immediately returned to the original gage at a rate of 1.0 mm/s. Force (N) and displacement (mm) data are continuously collected at 100 Hz throughout the test.

Load a dry test specimen such that it spans the two lower bars and is centered under the upper bar, with its sides parallel to the bars. For MD bending, the MD direction of the test specimen is perpendicular to the length of the 3 bars. Start the test and continuously collect force and displacement data.

Construct a graph of force (N) versus displacement (mm). From the graph, determine the maximum peak force and record as dry MD peak load to the nearest 0.01 N, denoting the test location as first or second absorbent zone. Now calculate the maximum slope of the curve between initial force and maximum force (during the loading portion of the curve) and record to the nearest 0.1 unit. Calculate the modulus as follows, and record as dry MD modulus to the nearest 0.001 N/mm², denoting the test location as first or second absorbent zone.

$$MD Dry Bending Modulus ( N / {mm}^{2} ) = ( Slope \times ( {Span}^{3} ) ) / ( 4 \times specimen width \times ( specimen {caliper}^{3} ) )$$

Calculate bending stiffness as follows, and record as dry MD bending stiffness to the nearest 0.1 N mm², denoting the test location as first or second absorbent zone.

In like fashion, the procedure is repeated for all five replicates of the dry test specimens from the first absorbent zone and all five replicates of the dry test specimens from the second absorbent zone. The arithmetic mean among the five replicate dry test specimens representing the first and second absorbent zones is calculated separately for each of the parameters and reported as Dry MD Peak Load to the nearest 0.01 N, Dry MD Bending Modulus to the nearest 0.001 N/mm², and Dry Bending Stiffness to the nearest N mm², with each parameter labeled appropriately to denote first or second absorbent zone test locations.

### Dry CD Ultra Sensitive 3 Point Bending Method

The CD (cross-direction) bending properties of a test specimen are measured using an ultra sensitive 3 point bend test on a universal constant rate of extension test frame (a suitable instrument is the MTS Alliance using TestSuite Software, as available from MTS Systems Corp., Eden Prairie, MN, or equivalent) equipped with a load cell appropriate for the forces being measured. The intention of this method is to mimic deformation created in the x-y plane by a wearer of an absorbent article during normal use. All testing is performed in a room controlled at 23° C.±3C and 50%±2% relative humidity and test samples are conditioned in this environment for at least 2 hours prior to testing.

The ultra sensitive 3 point bend method is designed to maximize the force signal to noise ratio when testing materials with very low bending forces. The force signal is maximized by using a high sensitivity load cell (e.g., 5 N), using a small span (load is proportional to the span cubed) and using a wide specimen width (total measured load is directly proportional to width). The fixture is designed such that the bending measurement is performed in tension, allowing the fixture mass to be kept to a minimum. Noise in the force signal is minimized by holding the load cell stationary to reduce mechanical vibration and inertial effect and by making the mass of the fixture attached to the load cell as low as possible.

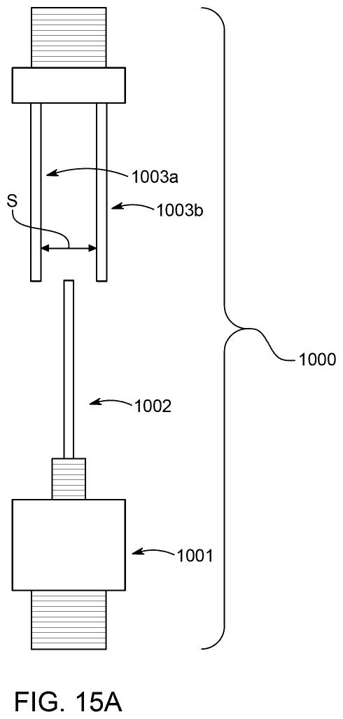

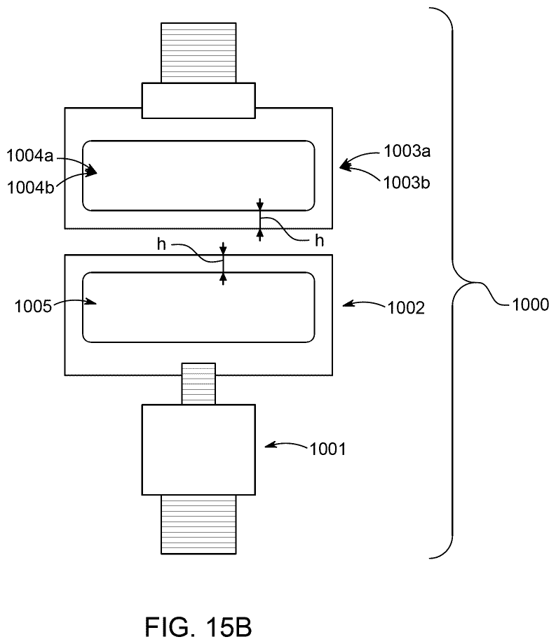

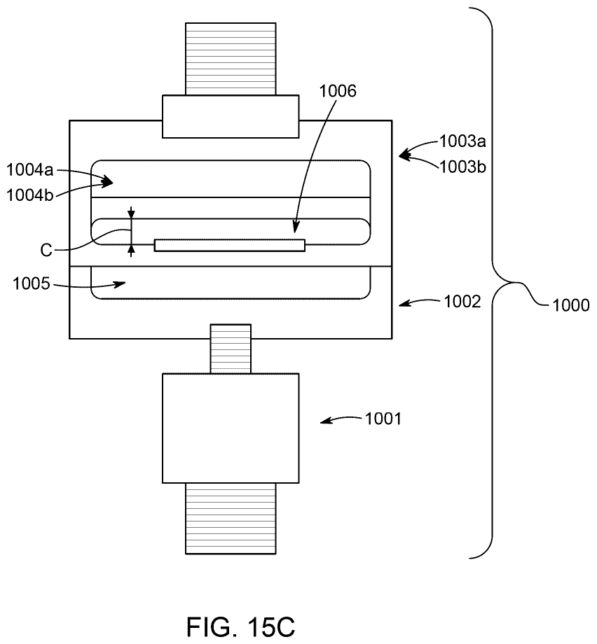

Referring to FIG. 15A-C, the load cell 1001 is mounted on the stationary crosshead of the universal test frame. The ultra sensitive fixture 1000 consists of three thin blades constructed of a lightweight, rigid material (such as aluminum, or equivalent). Each blade has a thickness of 1.0 mm, rounded edges and a length that is able to accommodate a bending width of 100 mm. Each of the blades has a cavity 1004a and 1004b (outside blades) and 1005 (central blade) cut out to create a height, h, of 5 mm of blade material along their horizontal edges. The two outside blades 1003a and 1003b are mounted horizontally to the moveable crosshead of the universal test frame, aligned parallel to each other, with their horizontal edges vertically aligned. The span, s, between the two outside blades 1003a and 1003b is 5 mm±0.1 mm (inside edge to inside edge). The central blade 1002 is mounted to the load cell on the stationary crosshead of the universal test frame. When in place, the central blade 1002 is parallel to the two outside blades 1003a and 1003b and centered at the midpoint between the outside blades 1003a and 1003b. The blade fixtures include integral adapters appropriate to fit the respective positions on the universal test frame and lock into position such that the horizontal edges of the blades are orthogonal to the motion of the crossbeam of the universal test frame.

Measurements are made on test specimens taken from rolls or sheets of the raw material, or test specimens obtained from a material layer removed from an absorbent article. When excising the material layer from an absorbent article, use care to not impart any contamination or distortion to the layer during the process. The excised layer should be free from residual adhesive and any fibers that may have transferred from underlying layers. To ensure that all adhesive and any transferred fibers are removed, soak the layer in a suitable solvent that will dissolve the adhesive and release any transferred fibers present without adversely affecting the material itself. One such solvent is THF (tetrahydrofuran, CAS 109-99-9, for general use, available from any convenient source). After the solvent soak, the material layer is allowed to thoroughly air dry in such a way that prevents undue stretching or other deformation of the material. After the material has dried, a test specimen is obtained as follows. The test specimen is cut from an area on the test material that is free of any folds or wrinkles. The dry specimens are prepared for CD bending (i.e., bending normal to the lateral axis of the sample) by cutting them to a width of 50.0 mm along the CD (cross direction; parallel to the lateral axis of the sample) and a length of 100.0 mm along the MD (machine direction; parallel to the longitudinal axis of the sample), maintaining their orientation after they are cut and marking the body-facing surface (or the surface intended to face the body of a finished article). In like fashion, five replicate dry test specimens are prepared.

The universal test frame is programmed such that the moveable crosshead is set to move in a direction opposite of the stationary crosshead at a rate of 1.0 mm/s. Crosshead movement begins with the specimen 1006 lying flat and undeflected on the outer blades 1003a and 1003b, continues with the inner horizontal edge of cavity 1005 in the central blade 1002 coming into contact with the top surface of the specimen 1006, and further continues for an additional 4 mm of crosshead movement. The crosshead stops at 4 mm and then immediately returns to zero at a speed of 1.0 mm/s. Force (N) and displacement (mm) are collected at 50 Hz throughout.

Prior to loading the test specimen 1006, the outside blades 1003a and 1003b are moved towards and then past central blade 1002 until there is approximately a 3 mm clearance, C, between the inner horizontal edges of cavities 1004a and 1004b in the outside blades 1003a and 1003b and the inner horizontal edge of cavity 1005 in the central blade 1002 (see FIG. 15C). The specimen 1006 is placed within clearance C such that it spans the inner horizontal edges of cavities 1004a and 1004b in the outside blades 1003a and 1003b, oriented such that the MD (short side) of the specimen is perpendicular to the horizontal edges of the blades and the body-facing surface of the specimen is facing up. Center the specimen 1006 between the outside blades 1003a and 1003b. Slowly move the outside blades 1003a and 1003b in a direction opposite of the stationary crosshead until the inner horizontal edge of cavity 1005 in the central blade 1002 touches the top surface of the specimen 1006. Start the test and continuously collect force and displacement data.

Force (N) is plotted versus displacement (mm). The maximum peak force is recorded to the nearest 0.001 N. The area under the curve from load onset up to the maximum peak force is calculated and recorded as bending energy to the nearest 0.001 N*mm. The recovery energy is calculated as the area under the curve where the force is unloaded from the maximum peak to 0.0 N and recorded as recovery energy to the nearest 0.001 N*mm. In like fashion, repeat the entire test sequence for a total of five dry test specimens and five wet test specimens.

For each test specimen, the arithmetic mean of the maximum peak force among like specimens is calculated to the nearest 0.001 N and recorded as Dry Peak Load. For each test specimen, the arithmetic mean of bending energy among like specimens is calculated to the nearest 0.001 N*mm and reported as Dry Bending Energy. For each test specimen, the arithmetic mean of recovery energy among like specimens is calculated to the nearest 0.001 N*mm and reported as Dry Recovery Energy.

### CD Cyclic Elongation to 3% Strain Method

The cyclic tensile and recovery response of absorbent article specimens are measured for ten cycles of load application (“elongation”) and load removal (“recovery”) using a universal constant rate of extension test frame. The test specimen is cycled ten times to 3% engineering strain, then back to zero engineering strain. For each cycle, stiffness, peak load, normalized energy to peak, normalized recovery energy, strain at start of cycle, and strain at end of cycle (i.e., “permanent strain”) are calculated and reported. The intention of this method is to understand the ability of samples to stretch in the x-y plane as a result of bodily forces, and then recover to their original state. All measurements are performed in a laboratory maintained at 23° C.±2 C.° and 50%±2% relative humidity and test specimens are conditioned in this environment for at least 2 hours prior to testing.

A suitable universal constant rate of extension test frame is the MTS Alliance interfaced to a computer running TestSuite control software (available from MTS Systems Corp, Eden Prairie, MN), or equivalent. The universal test frame is equipped with a load cell for which forces measured are within 1% to 99% of the limit of the cell. The fixtures used to grip the test specimen are lightweight (<80 grams), vise action clamps with knife or serrated edge grip faces that are at least 40 mm wide. The fixtures are installed on the universal test frame and mounted such that they are horizontally and vertically aligned with one another.

Measurements are made on test specimens taken from rolls or sheets of the raw material, or test specimens obtained from a material layer removed from an absorbent article. When excising the material layer from an absorbent article, use care to not impart any contamination or distortion to the layer during the process. The excised layer should be free from residual adhesive and any fibers that may have transferred from underlying layers. To ensure that all adhesive and any transferred fibers are removed, soak the layer in a suitable solvent that will dissolve the adhesive and release any transferred fibers present without adversely affecting the material itself. One such solvent is THF (tetrahydrofuran, CAS 109-99-9, for general use, available from any convenient source). After the solvent soak, the material layer is allowed to thoroughly air dry in such a way that prevents undue stretching or other deformation of the material. After the material has dried, a test specimen is obtained. The test specimen is cut from an area on the test material that is free of any residual of folds or wrinkles. The test specimen is as long as the lateral length of the article (parallel to the lateral axis of the article, or the intended lateral axis of the article). When excising specimens from absorbent articles of different sizes and widths, the total specimen length (Lₜₒₜₐₗ) may vary from product to product, thus the results will be normalized to compensate for this variation. The test specimen has a width of 25.4 mm wide (parallel to the longitudinal axis, or intended longitudinal axis of the article). Specimen width (w)=25.4 mm. Measure and record the total specimen length (Lₜₒₜₐₗ) to the nearest 0.1 mm. In like fashion, five replicate test specimens are prepared.

Measure the thickness (t) of the test specimen using a manually-operated micrometer equipped with a pressure foot capable of exerting a steady pressure of 0.1 psi±0.01 psi. The manually-operated micrometer is a dead-weight type instrument with readings accurate to 0.01 mm. A suitable instrument is Mitutoyo Series 543 ID-C Digimatic, available from VWR International, or equivalent. The pressure foot is a flat circular moveable face with a diameter no greater than 25.4 mm. The test specimen is supported by a horizontal flat reference platform that is larger than and parallel to the surface of the pressure foot. Zero the micrometer against the horizontal flat reference platform. Place the test specimen onto the platform, centered beneath the pressure foot. The pressure foot is lowered by hand with a descent rate of 3±1 mm/s until the full weight of the pressure is exerted onto the specimen. After 5 seconds elapse, the thickness is recorded as specimen thickness (t) to the nearest 0.01 mm.

Prepare the universal test frame as follows. Set the initial grip to grip separation distance to a nominal gage length (Lₙₒₘᵢₙₐₗ) that is shorter than the total specimen length and such that the specimen can be gripped securely at both ends (i.e., Lₙₒₘᵢₙₐₗ<Lₜₒₜₐₗ). Then zero the crosshead. Program the test frame to move the grips closer together by an intentional slack of 1 mm to ensure no pretension force exists on the test specimen at the onset of the test. (During this motion, the specimen will become slack between the tensile grips.) Next, the grips will move apart at a slack speed of 1 mm/s until the slack preload of 0.05 N is exceeded. At this point, the following are true. 1) The crosshead position signal (mm) is defined as the specimen slack (L_{slack}). 2) The initial specimen gage length (L₀) is calculated as the nominal gage length plus the slack L₀=Lₙₒₘᵢₙₐₗ+L_{slack}, where units are in millimeters. 3) The crosshead extension (ΔL) is set to zero (0.0 mm). 4) The crosshead displacement (mm) is set to zero (0.0 mm). At this position the engineering strain is zero, 0.0. Engineering strain is calculated as the change in length (ΔL) divided by the initial length (L₀). Engineering strain=ΔL/L₀. For one test cycle, the grips move apart at the initial speed of 1 mm/s until the engineering strain endpoint of 0.03 mm/mm is exceeded, immediately followed by the grips moving toward each other at the initial speed of 1 mm/s until the crosshead signal becomes less than the crosshead return position of 0 mm. The test cycle is repeated until a total of 10 cycles is complete.

The test is executed by inserting the test specimen into the grips such that the long axis of the specimen is parallel and centered with the motion of the crosshead. Start the test and continuously collect time, force and displacement data at a data acquisition rate of 100 Hz.

Construct a graph of load (N) versus displacement for all ten cycles. For each cycle, perform the following. Record peak load to the nearest 0.01 N. Calculate the energy to peak (Eₚₑₐₖ) as the area under the load versus displacement curve from the cycle start to the strain endpoint of 0.03 mm/mm (during the loading portion of the cycle) and record to the nearest 0.01 N*mm. Calculate the return energy (Eᵣₑₜᵤᵣₙ) as the area under the load versus displacement curve from the strain endpoint of 0.03 mm/mm to the crosshead return of 0 mm (during the unloading portion of the cycle) and record as recovery energy to the nearest 0.01 N*mm. Calculate the normalized energy to peak (NEₚₑₐₖ) as the energy to peak divided by the initial length, where NEₚₑₐₖ=Eₚₑₐₖ/L₀, and record to the nearest 0.01 mN. Calculate the normalized return energy (NEᵣₑₜᵤᵣₙ) as the return energy divided by the initial length (NEᵣₑₜᵤᵣₙ=Eᵣₑₜᵤᵣₙ/L₀), and record to the nearest 0.01 mN. Units of NEₚₑₐₖ and NEᵣₑₜᵤᵣₙ are milliNewtons (mN).

Now construct a graph of engineering stress (a) versus engineering strain for all ten cycles, and for each cycle perform the following. Engineering stress, in units of N/mm², is the load divided by the cross sectional area of the specimen, where the cross sectional area is the specimen width (w) multiplied by the thickness (t), σ=Load/(w*t). Determine the modulus, or slope of the stress versus strain curve for a line between the point that occurs at the minimum force and the point that occurs at the maximum force (during the loading portion of the cycle) and record as modulus to the nearest 0.01 N/mm². Calculate stiffness by multiplying the modulus by the specimen thickness and record as tensile stiffness to the nearest 0.01 N/mm. The strain of the test specimen at the beginning of the cycle is defined by the strain when the slack preload of 0.05 N is exceeded for that cycle (during the loading portion of the cycle), and is recorded as cycle initial strain to the nearest 0.01 mm/mm. The strain of the test specimen at the end of the cycle is defined by the strain when the load becomes less than the preload of 0.05 N for that cycle (during the unloading portion of the cycle), and is recorded as permanent strain to the nearest 0.01 mm/mm. In like fashion, the overall procedure is now repeated for all five replicates.

The arithmetic mean among the five replicate test specimens is calculated for each of the parameters, for each of the ten cycles, and reported as Peak Load to the nearest 0.01 N, Normalized Energy to Peak to the nearest 0.01 mN, Normalized Recovery Energy to the nearest 0.01 mN, Tensile Stiffness to the nearest 0.01 N/mm, Cycle Initial Strain to the nearest 0.01 mm/mm, and Permanent Strain to the nearest 0.001 mm/mm.

### Structural Bond Sites Pattern Spacing and Area Measurement Method

The spacing between the discreet structural bond sites that are used to create a quilt-like pattern on absorbent article samples, and the overall area taken up by the sum of those elements in a specified region of the sample are measured on images of the absorbent article sample acquired using a flatbed scanner. The scanner is capable of scanning in reflectance mode at a resolution of 2400 dpi and 8 bit grayscale. A suitable scanner is an Epson Perfection V750 Pro from Epson America Inc., Long Beach CA, or equivalent. The scanner is interfaced with a computer running an image analysis program. A suitable program is ImageJ v. 1.52, National Institute of Health, USA, or equivalent. The sample images are distance calibrated against an acquired image of a ruler certified by NIST. To enable maximum contrast, the sample is backed with an opaque, black background of uniform color prior to acquiring the image. To prevent overly saturated images, a neutral density filter can be placed between the specimen and the scanner glass. All testing is performed in a conditioned room maintained at about 23±2° C. and about 50±2% relative humidity.

The test sample is prepared as follows. Remove the absorbent article from any wrapper present. If the article is folded, gently unfold it and smooth out any wrinkles. If wings are present, extend them but leave the release paper intact. The test samples are conditioned at about 23° C.±2 C.° and about 50%±2% relative humidity for 2 hours prior to testing.

Images are obtained as follows. The ruler is placed on the scanner bed such that it is oriented parallel to the sides of the scanner glass. An image of the ruler (the calibration image) is acquired in reflectance mode at a resolution of 2400 dpi (approximately 94 pixels per mm) and in 8-bit grayscale. The calibration image is saved as an uncompressed TIFF format file. After obtaining the calibration image, the ruler is removed from the scanner glass and the test sample is scanned under the same scanning conditions as follows. Place the test sample onto the center of the scanner glass and secure, if necessary, such that it lies flat with the body-facing surface of the sample facing the scanner's glass surface. The sample is oriented in such a way that the entire sample is within the glass surface. The black background is placed on top of the sample, the scanner lid is closed, and a scanned image of the entire sample is acquired with the same settings as used for the calibration image. As previously mentioned, to reduce overly saturated images, a neutral density filter can be placed between the sample and the scanner glass. The sample image is saved as an uncompressed TIFF format file.

The sample image is analyzed as follows. Open the calibration image file in the image analysis program, and calibrate the image resolution using the imaged ruler to determine the number of pixels per millimeter. Now open the sample image in the image analysis program, and set the distance scale using the image resolution determined from the calibration image. Now visually inspect the pattern of emboss elements present on the sample in the image and identify the zones of the pattern that are to be analyzed. For example the absorbent article can be divided into three equal lengths zones in the machine direction such as the front one third zone, zone 1, the central one third zone, zone 2 and the end one third zone, zone 3 as example. Use the image analysis tools to draw a shape along the outer perimeter of the first discreet zone to be analyzed. Measure the area of this first zone and record as Zone 1 Total Area to the nearest 0.01 mm². Now measure the area of each individual, discreet emboss element that lies inside of the zone 1 perimeter as follows. Draw a minimum bounding circle around an individual emboss element such that no portion of the emboss element lies outside of the bounding circle. Now measure the area of the bounding circle for that emboss element and record the emboss element area to the nearest 0.01 mm². In like fashion, measure the area of every emboss element, including portions of emboss elements, that lie inside zone 1 and record each to the nearest 0.01 mm². Now sum the areas of all of the emboss elements inside of zone 1 and record as Zone 1 Total Emboss Element Area to the nearest 0.01 mm². Divide the Zone 1 Total Emboss Element Area by the Zone 1 Total Area then multiply by 100 and record as Zone 1% Total Area Represented by Emboss Elements. The spacing between each discreet emboss element inside of zone 1 is measured as follows. Measure the distance from the center of the minimum bounding circle drawn around a discreet emboss element inside of zone 1, as described herein, to the center of the minimum bounding circle drawn around the nearest neighboring discreet emboss element inside of zone 1, and record this distance as emboss spacing to the nearest 0.01 mm. In like fashion, repeat for all neighboring emboss elements inside of zone 1, and record each distance to the nearest 0.01 mm. Now calculate the arithmetic mean among all measured emboss spacings measured between nearest neighbors inside of zone 1, and record as Zone 1 Emboss Spacing to the nearest 0.01 mm.

In like fashion, the entire procedure is repeated for each additional zone containing emboss elements that is present on the test sample and label accordingly as Zone 2, Zone 3, etc.

### Nonwoven Thickness—Pressure Method

The thickness of a test specimen is measured as the distance between a reference platform on which the specimen rests and a pressure foot that exerts a specified amount of pressure onto the specimen over a specified amount of time. For purposes herein, thickness is measured and reported at two different confining pressures (7 g/cm² and 70 g/cm²). All measurements are performed in a laboratory maintained at 23° C.±2 C.° and 50%±2% relative humidity and test specimens are conditioned in this environment for at least 2 hours prior to testing.

Thickness is measured with a manually-operated micrometer equipped with a pressure foot capable of exerting a steady pressure (7 g/cm² and 70 g/cm²) onto the test specimen. The manually-operated micrometer is a dead-weight type instrument with readings accurate to 0.01 mm. A suitable instrument is Mitutoyo Series 543 ID-C Digimatic, available from VWR International, or equivalent. The pressure foot is a flat ground circular movable face with a diameter that is smaller than the test specimen and capable of exerting the required pressure. A suitable pressure foot has a diameter of 25.4 mm, however a smaller or larger foot can be used depending on the size of the specimen being measured. The test specimen is supported by a horizontal flat reference platform that is larger than and parallel to the surface of the pressure foot. The system is calibrated and operated per the manufacturer's instructions.

Measurements are made on test specimens taken from rolls or sheets of the raw material, or test specimens obtained from a material layer removed from an absorbent article. When excising the material layer from an absorbent article, use care to not impart any contamination or distortion to the layer during the process. The excised layer should be free from residual adhesive and any fibers that may have transferred from underlying layers. To ensure that all adhesive and any transferred fibers are removed, soak the layer in a suitable solvent that will dissolve the adhesive and release any transferred fibers present without adversely affecting the material itself. One such solvent is THF (tetrahydrofuran, CAS 109-99-9, for general use, available from any convenient source). After the solvent soak, the material layer is allowed to thoroughly air dry in such a way that prevents undue stretching or other deformation of the material. After the material has dried, a test specimen is obtained from an area free of folds or wrinkles, and it must be larger than the pressure foot.

To measure thickness at a confining pressure of 7 g/cm², first zero the micrometer against the horizontal flat reference platform. Place the test specimen on the platform with the test location centered below the pressure foot. Gently lower the pressure foot with a descent rate of 3.0 mm±1.0 mm per second until the full pressure is exerted onto the test specimen. Wait 5 seconds and then record the thickness of the test specimen to the nearest 0.01 mm. In like fashion, repeat for a total of ten replicate test specimens. Calculate the arithmetic mean for all thickness measurements obtained at a confining pressure of 7 g/cm² and report as Thickness at 7 g/cm² to the nearest 0.01 mm.

To measure thickness at a confining pressure of 70 g/cm², first zero the micrometer against the horizontal flat reference platform. Place the test specimen on the platform with the test location centered below the pressure foot. Gently lower the pressure foot with a descent rate of 3.0 mm±1.0 mm per second until the full pressure is exerted onto the test specimen. Wait 5 seconds and then record the thickness of the test specimen to the nearest 0.01 mm. In like fashion, repeat for a total of ten replicate test specimens. Calculate the arithmetic mean for all thickness measurements obtained at a confining pressure of 70 g/cm² and report as Thickness at 70 g/cm² to the nearest 0.01 mm.

### Dimensional and Geometric Measurement Method

Simple dimensional and geometric measurements like width, length, area, angles, and radius of curvature of specified locations (described herein) on the surface of an absorbent article are measured on images of the absorbent article sample (or prepared test specimen of a given zone) acquired using a flatbed scanner. The scanner is capable of scanning in reflectance mode at a resolution of 2400 dpi and 8 bit grayscale. A suitable scanner is an Epson Perfection V750 Pro from Epson America Inc., Long Beach CA, or equivalent. The scanner is interfaced with a computer running an image analysis program. A suitable program is ImageJ v. 1.52, National Institute of Health, USA, or equivalent. The sample images are distance calibrated against an acquired image of a ruler certified by NIST. To enable maximum contrast, the sample is backed with an opaque, black background of uniform color prior to acquiring the image. To prevent overly saturated images, a neutral density filter can be placed between the specimen and the scanner glass. All testing is performed in a conditioned room maintained at about 23±2° C. and about 50±2% relative humidity.

The test sample is prepared as follows. Remove the absorbent article from any wrapper present. If the article is folded, gently unfold it and smooth out any wrinkles. If wings are present, extend them but leave the release paper intact. In like fashion, a total of five replicate intact test samples are prepared. The test samples are conditioned at about 23° C.±2 C.° and about 50%±2% relative humidity for 2 hours prior to testing. To note, the area of test specimens prepared as specified in the basis weight section of the Absorbent Article Caliper, Basis Weight, and Density Method, described herein, is also measured using this imaging technique, and no further preparation of those test specimens is required.

Images are obtained as follows. The ruler is placed on the scanner bed such that it is oriented parallel to the sides of the scanner glass. An image of the ruler (the calibration image) is acquired in reflectance mode at a resolution of 2400 dpi (approximately 94 pixels per mm) and in 8-bit grayscale. The calibration image is saved as an uncompressed TIFF format file. After obtaining the calibration image, the ruler is removed from the scanner glass and the test sample, or test specimen, is scanned as follows. Place the test sample, or prepared test specimen, onto the center of the scanner glass and secure, if necessary, such that it lies flat with the body-facing surface of the test sample, or prepared test specimen, facing the scanner's glass surface. The test sample, or prepared test specimen, is oriented in such a way that the entire test sample, or prepared test specimen, is within the glass surface. The black background is placed on top of the test sample, or prepared test specimen, the scanner lid is closed, and a scanned image of the entire test sample, or prepared test specimen, is acquired with the same settings as used for the calibration image. As previously mentioned, to reduce overly saturated images, a neutral density filter can be placed between the sample and the scanner glass. The test sample, or prepared test specimen image is saved as an uncompressed TIFF format file.

The test sample image is analyzed to make width, length, angular, and radius of curvature measurements as follows. Open the calibration image file in the image analysis program, and calibrate the image resolution using the imaged ruler to determine the number of pixels per millimeter. Now open the test sample image in the image analysis program, and set the distance scale using the image resolution determined from the calibration image. Linear measurements are made using the line measurement tool within the image analysis software. As depicted in FIG. 2B, FIG. 5A, FIG. 5C, FIG. 5D, FIG. 6A-C, FIG. 7A, FIG. 7B, widths WS, Mc, Wm, Wn, Wp, Mg, and Tz and lengths De, Lp, Px, Pc, Ls, Lm, Lz, Df, and Dr are measured using the locations for each dimension as specified herein. Angular measurements between specified regions of the absorbent article are made using the angle tool within the image analysis software and recorded to the nearest degree. A radius of curvature is the radius of a circle which touches a curve at a given point and has the same tangent and curvature at that point. Using the shape tools within the image analysis software, a circle is fit within a given region as specified herein (e.g. Rc), and the radius of that circle is measured and recorded to the nearest 0.1 mm. A total of five replicate test samples are imaged and analyzed in like fashion, and each parameter is recorded. The arithmetic mean among the five replicate test samples is calculated for each of the parameters measured and linear measurements are reported to the nearest 0.1 mm, angular measurements are reported to the nearest degree, and radius of curvature is reported to the nearest 0.1 mm.

The test specimen images of the first absorbent zone and second absorbent zone are analyzed as follows. Open the calibration image file in the image analysis program, and calibrate the image resolution using the imaged ruler to determine the number of pixels per millimeter. Now open the test specimen image in the image analysis program, and set the distance scale using the image resolution determined from the calibration image. Area measurements are made using the freehand selection tool, or equivalent, within the image analysis software to draw a shape that runs along the outer perimeter of the prepared test specimen. Now measure the area of the drawn shape and record as area to the nearest 0.01 mm², denoting the zone as either first or second absorbent zone, and also denoting the corresponding sample number. In like fashion, repeat until the area of each of the five replicate test specimens from each zone (first and second absorbent zones) is measured and recorded, denoting the corresponding sample number for each replicate. Now proceed back to the basis weight portion of the Absorbent Article Caliper, Basis Weight, and Density Method.

### Absorbent Article Caliper, Basis Weight, and Density Method

The caliper, basis weight, and density method specifies how these parameters are measured for two different test locations on an absorbent article sample. Referring to FIG. 5A, the test locations include the central absorbent zone 306 and the outer absorbent zone 325. All testing is performed in a room controlled at 23° C.±3° C. and 50%±2% relative humidity.

The absorbent article test samples are conditioned at 23° C.±3C and 50%±2% relative humidity two hours prior to testing. Remove the test sample from its outer wrapper, then remove the protective cover/release paper from the undergarment fastening adhesive on the garment facing side of the sample. Lightly dust the undergarment fastening adhesive with talc powder to mitigate any tackiness. In like fashion, a total of five replicate test samples are prepared. The test samples are labeled consecutively as sample 1 through sample 5 by marking a small number on the backsheet/garment side of each sample.

### Absorbent Article Caliper

Caliper (or “thickness”) is measured at specified test locations on the absorbent article sample using a manually-operated micrometer equipped with a pressure foot capable of exerting a steady pressure of 7 g/cm². The manually-operated micrometer is a dead-weight type instrument with readings accurate to 0.01 mm. A suitable instrument is Mitutoyo Series 543 ID-C Digimatic, available from VWR International, or equivalent. The pressure foot is a flat circular moveable face with a diameter that is smaller than the test region being measured. The test sample is supported by a horizontal flat reference platform that is larger than and parallel to the surface of the pressure foot. Zero the micrometer against the horizontal flat reference platform. Place the test sample onto the platform, with the test location centered beneath the pressure foot. The pressure foot is lowered by hand with a descent rate of 3±1 mm/s until the full weight of the pressure is exerted onto the sample. After 5 seconds elapse, the thickness is recorded as absorbent article caliper to the nearest 0.01 mm, denoting the test location as either first or second absorbent zone, and also denoting the sample number, as previously marked. As specified, the caliper is measured at three separate, non-overlapping regions within the first absorbent zone 306 of each intact absorbent article sample. The arithmetic mean of the caliper values collected for the first absorbent zone 306 across all five replicate samples is calculated and reported as Absorbent Article Caliper in the First Absorbent Zone to the nearest 0.01 mm. Now the caliper is measured at three separate, non-overlapping regions within the second absorbent zone 325 of each intact absorbent article sample, avoiding any portion of the perimeter seal 230. The arithmetic mean of the caliper values collected for the second absorbent zone 325 across all five replicate samples is calculated and reported as Absorbent Article Caliper in the Second Absorbent Zone to the nearest 0.01 mm. Proceed to the basis weight measurement using the same prepared and numbered test samples.

### Absorbent Article Basis Weight

The basis weight of the first absorbent zone 306 and the second absorbent zone 325 are measured separately for each prepared test sample using specially machined cutting dies. A first cutting die is used to excise a specimen from the region of the article that comprises the second absorbent zone 325 inbound of the perimeter seal 230. The die is prepared such that the cutting blades form a circle or square as large as possible that fits within the rear region 23 of the article, inbound of the perimeter seal 230. The test specimen of the second absorbent zone does not include any portion of the first absorbent zone 306 or any flex bond channel regions 160. A second cutting die in the exact shape of the first absorbent zone is prepared such that the cutting line of the die is aligned with the exact perimeter of the entire region designated as the first absorbent zone 306, as depicted in FIG. 5A. It is understood that for each unique product design, and for every different product size, a set of specially machined cutting dies must be prepared. Test specimens from the first absorbent zone and the second absorbent zone are punched out and corresponding sample number is labeled on the backsheet/garment side of each of these prepared test specimens. Careful handling of the test specimens is required to prevent the loss of any particles or material fibers contained within each. Record the mass of the specimen that is the first absorbent zone, and record as absorbent article first absorbent zone mass to the nearest 0.001 g, denoting the sample number as well. Now record the mass of the specimen that is the second absorbent zone, and record as absorbent article second absorbent zone mass to the nearest 0.001 g, denoting the sample number as well. In like fashion, punch out the first and second absorbent zones, labeling the corresponding sample number on each, for the remaining four replicate test samples. Record the mass of each zone to the nearest 0.001 g, denoting the zone as either absorbent article first or second absorbent zone, and also denoting the corresponding sample number. Now proceed to the Dimensional and Geometric Measurement Method, as described herein, to measure the area of each of the test specimens representing the absorbent article first and second absorbent zones. After obtaining the area of each zone, calculate the basis weight by dividing the mass (g) by the area (converted to square meters), and record as absorbent article basis weight to the nearest 0.1 g/m², denoting the zone as either first or second absorbent zone, and also denoting the corresponding sample number. Calculate the arithmetic mean of the basis weight across all five test specimens of the first absorbent zone, and report as Absorbent Article Basis Weight of the First Absorbent Zone to the nearest 0.1 g/m². Calculate the arithmetic mean of the basis weight across all five test replicates of the second absorbent zone, and report as Absorbent Article Basis Weight of the Second Absorbent Zone to the nearest 0.1 g/m². Retain all of these prepared test specimens of the absorbent article first and second absorbent zones as they will be used for the Basis Weight of the Inner Core Layer method, as described herein.

### Absorbent Article Density

The density of the first absorbent zone 306 and the second absorbent zone 325 are calculated as follows. For the first absorbent zone test specimen from Sample 1, divide the basis weight in g/m² (absorbent article basis weight, first absorbent zone, sample 1) by the caliper, mm (absorbent article caliper, first absorbent zone, sample 1), then divide the quotient by 1000 and record as absorbent article density of the first absorbent zone to the nearest 0.001 g/m³. In like fashion, calculate density of the first absorbent zone for all five replicate test specimens. Now calculate the arithmetic mean of density across all five test specimens of the first absorbent zone, and report as Absorbent Article Density of the First Absorbent Zone to the nearest 0.001 g/m³. For the second absorbent zone test specimen from Sample 1, divide the basis weight in g/m² (absorbent article basis weight, second absorbent zone, sample 1) by the caliper, mm (absorbent article caliper, second absorbent zone, sample 1), then divide the quotient by 1000 and record as absorbent article density of the second absorbent zone to the nearest 0.001 g/m³. In like fashion, calculate density of the second absorbent zone for all five replicate test specimens. Now calculate the arithmetic mean of density across all five test specimens of the second absorbent zone, and report as Absorbent Article Density of the Second Absorbent Zone to the nearest 0.001 g/m³.

### Inner Core Layer Basis Weight

The basis weight of the inner core layer, at the points in the first absorbent zone and the second absorbent zone will typically be known by the manufacturer from the product making specification. However, if the basis weight is not known for a given article, the basis weight can be measured in the following manner.

The basis weight of the inner core layer within the first absorbent zone 306 and the second absorbent zone 325 are measured separately for each of the retained absorbent article test specimens that were prepared in the basis weight section of the Absorbent Article Caliper, Basis Weight, and Density Method, as described herein. There will be five absorbent article test specimens that are the first absorbent zone (labeled as samples 1-5) and five absorbent article test specimens that are the second absorbent zone (also labeled as samples 1-5). To note, the measured area values for each test specimen that was previously recorded to calculate the basis weight of the absorbent article first and second absorbent zones in the basis weight section of the Absorbent Article Caliper, Basis Weight, and Density Method will also be used to calculate the basis weight of the inner core layer from each of these zones.

The inner core layer is removed from the test specimens, the mass recorded, and the basis weight calculated as follows. Begin with the absorbent article test specimen of the first absorbent zone from sample 1. Carefully remove the topsheet layer, backsheet film, the upper nonwoven layer and the lower nonwoven layer from the inner core layer of the test specimen, ensuring that in the process no particles or fibers are lost from the inner core layer. It is understood that, depending on the unique structure of the absorbent article, additional layers may need to be removed from the absorbent article test specimen in order to obtain a final test specimen that comprises only the inner core layer. Record the mass of the inner core layer test specimen to the nearest 0.001 g, denoting first absorbent zone, sample 1. Divide the mass of the inner core layer from the first absorbent zone of sample 1 by the area of the absorbent article first absorbent zone of sample 1 (previously measured), and record as basis weight of the inner core layer, denoting first absorbent zone, sample 1, to the nearest 0.1 g/m². In like fashion, repeat the procedure until the basis weight of the inner core layer from the first absorbent zone is measured and recorded for each of the five first absorbent zone test specimen replicates from samples 1 through 5. Calculate the arithmetic mean across the basis weight values obtained for all five first absorbent zone replicates, and report as Inner Core Layer Basis Weight of the First Absorbent Zone to the nearest 0.1 g/m². In like fashion, this entire procedure is repeated for the five absorbent article test specimens from the second absorbent zone of samples 1 through 5, and the arithmetic mean across the five basis weight values is calculated and reported as Inner Core Layer Basis Weight of the Second Absorbent Zone to the nearest 0.1 g/m².

### Z-Compression Method

The Z-compression method measures the compression behavior along the z-direction of a test specimen, on a Constant Rate of Extension (CRE) universal mechanical test system using a load cell for which the forces measured are within 1% to 99% of the limit of the cell (preferably 100 N). A suitable instrument is the MTS Alliance using TestSuite Software, as available from MTS Systems Corp., Eden Prairie, MN, or equivalent. All testing is performed in a room controlled at 23° C.±3° C. and 50%±2% relative humidity.

The upper and lower fixtures of the test system are circular parallel plate compression platens made of stainless steel. The platen mounted on the moveable CRE fixture has a diameter of 12.5 mm, and the platen mounted on the stationary CRE fixture has a diameter >12.5 mm. Both platens have adapters compatible with the mounts of the CRE test machine, capable of securing the platens with their opposing surfaces lying along parallel planes that are orthogonal to the motion of the crossbeam of the CRE test machine.

The absorbent article samples are conditioned at 23° C.±3° C. and 50%±2% relative humidity two hours prior to testing. Remove the test sample from its outer wrapper, then remove the protective cover/release paper from the undergarment fastening adhesive on the garment facing side of the sample. Lightly apply talc powder to the adhesive to mitigate any tackiness. To obtain a test specimen for measurement, a circular die with a diameter of 12.5 mm is used. Test specimens are obtained from two separate test locations on the absorbent article test sample from areas that are free from any folds or wrinkles, as follows. Referring to FIG. 5A, the first test specimen is obtained from the central zone 320 of the first absorbent zone 306 such that the test specimen is comprised of only the first absorbent zone and does not include any portion of a flex bond region or other absorbent zone. The second test specimen is obtained from the rear-most position possible (inbound of the perimeter seal 230) on the test sample within the rear region 23 that includes the second absorbent zone 325. The second test specimen does not include any portion of any flex bond channel region, structural bond site, or perimeter seal. In like fashion, five replicate test specimens are prepared from the central and outer absorbent zones on five replicate test samples.

Prepare the universal test frame for a compression test to measure force and distance for one cycle of loading (compression) and unloading (recovery) as follows. The crosshead motion is programmed such that the upper platen moves down with respect to the lower platen at a rate of 0.2 mm/s until an endpoint load of 0.846 N is reached, then the crosshead immediately returned to the original gauge (platen separation distance).

Execute the test as follows. Move the platens such that the initial distance between contact surfaces of the platens (gauge) is 25 mm, then zero the crosshead and load cell. Place the test specimen, with the wearer-facing surface upward, onto the bottom platen with the longitudinal and lateral midpoint of the test specimen centered under the upper platen. Manually adjust the position of the upper platen such that its contact surface is about 1 mm above the upper surface of the test specimen. Start the test and continuously collect force (N) and displacement (mm) data at a rate of 100 Hz.

Construct a graph of force (N) versus thickness (mm), across the array of data collected for the entire cycle. To note, at each datapoint, thickness is the original gauge (25 mm) minus the crosshead position (mm). From the resulting force (N) vs thickness (mm) curve, calculate the area under the loading (compression) portion of the curve from the initial thickness to the minimum thickness, and record as energy of z-compression to the nearest 0.1 N*mm, denoting the test location as central or outer absorbent zone.

In like fashion, repeat the procedure for all five replicate test specimens from the central absorbent zone and all five replicate test specimens from the outer absorbent zone. Calculate and report the arithmetic mean across all five replicates from each test location and report as Energy of Z-Compression to the nearest 0.1 N*mm, denoting central or outer absorbent zones.

### Micro-CT Measurement Method

The micro-CT measurement method is used to obtain images of the cross-section of a test specimen to enable visualization of the microstructure of an absorbent article, including the interconnectivity of layers within the article in specific regions of interest. These images enable qualitative and quantitative assessments to be made related to the proximity of adjacent layers within the test specimen, and the resultant size and shape of specified zones located within the test specimen. This method is based on analysis of a three-dimensional x-ray sample image obtained on a micro-CT instrument (a suitable instrument is the Scanco μCT 50 available from Scanco Medical AG, Switzerland, or equivalent). The micro-CT instrument is a cone beam microtomograph with a shielded cabinet. A maintenance free x-ray tube is used as the source with an adjustable diameter focal spot. The x-ray beam passes through the sample, where some of the x-rays are attenuated by the sample. The extent of attenuation correlates to the mass of material the x-rays have to pass through. The transmitted x-rays continue on to the digital detector array and generate a two-dimensional projection image of the sample. A three-dimensional image of the sample is generated by collecting several individual projection images of the sample as it is rotated, which are then reconstructed into a single three-dimensional image. The instrument is interfaced with a computer running software to control the image acquisition and reconstruction of the raw data into a three-dimensional image. The three-dimensional image is then analyzed using image analysis software (suitable image analysis software are MATLAB available from The Mathworks, Inc., Natick, MA, and Avizo 2022.2 available from Visualization Sciences Group/FEI Company, Burlington, MA, or equivalents) to identify specified zones with the test specimen, measure the distances between individual layers and zones, thickness of zones as well as any angle created as one zone transitions to another within the test specimen.

### Sample Preparation:

A test specimen is excised from the test sample using a very sharp blade as follows. The test specimen is taken from an area free of folds or wrinkles using care to not impart any contamination or distortion to the specimen during the preparation process. The test specimen is excised from a region of the test sample in such a way that it comprises a portion of both the first absorbent zone and the second absorbent zone, including any transition zone that may be present. The test specimen is roughly 90 mm in diameter. In like fashion, a total of 3 replicate test specimens are prepared from three different test samples. The test specimens are conditioned at about 23° C.±2 C.° and about 50%±2% relative humidity for 2 hours prior to testing.

### Image Acquisition:

The micro-CT instrument is set up and calibrated according to the manufacturer's specifications. The test specimen is placed on a low density foam and placed into the appropriate holder. This will allow the test specimen to lay horizontal and be scanned with minimal attenuation from any surrounding material. A single three-dimensional dataset of contiguous 13 μm (microns) isotropic voxels is collected. The three-dimensional data set has dimensions of 96.7 mm on each side in the XY-plane and a sufficient number of slices to fully include the entire Z-direction of the test specimen. Images are acquired with the source at 70 key and 114 μA with no additional low energy filter. These current and voltage settings may be optimized to produce the maximum contrast in the projection data with sufficient x-ray penetration through the test specimen, but once optimized held constant for all substantially similar test specimens. A total of 3000 projection images are obtained with a total integration of 500 msec integration time and 4 averages per projection. The projection images are reconstructed into a three-dimensional dataset having an isotropic spatial resolution of 13 μm (microns), and saved in 16-bit RAW format to preserve the full detector output signal for analysis. For optimal visualization purposes, the data was scaled to 8 bit using a scale factor of 0.4 and subsampled to 26 micron resolution.

### Image Processing:

The three-dimensional dataset is loaded into the image analysis software and trimmed (cropped) to a rectangular prism three-dimensional image of the analysis region by removing the surrounding holder and the low density mounting material from the three-dimensional dataset. Trimming is performed such that the maximum amount of the test specimen in the analysis region is retained in the three-dimensional image, and the empty space above and below the test specimen is minimized. Within the three-dimensional image, every 10 cross sectional slices are averaged together to create less noise. This averaging creates a thicker slice representing a 260 micron thick slab along the viewing direction. In-plane resolution is 26 microns.

The three-dimensional image is oriented so that the upper surface (topsheet, or body side of the test specimen) is as close to parallel with the XY-plane as possible. Now qualitative observations can be made regarding the proximity of adjacent layers present in the test specimen including interconnectivity between said layers and the overall shape of the various zones present (i.e. first and second absorbent zones and the transition between said zones). In addition to the qualitative observations, simple quantitative measures (e.g. thickness of zones, distance between zones, the angle formed as one zone transitions to the other, etc.) are possible using measurement tools available within the image analysis software.

### Flex Bond Channel Measurements

The flex bond channel width and the flex bond channel depth of the flex bond channel region 160 formed along the longitudinal axis in the rear region 23 of the absorbent article test sample are measured using optical profilometry to obtain the areal surface topography of the body facing side of the test sample. The flex bond channel width is measured at the base of the depressions, and the depth of the flex bond channel is measured relative to an adjacent, non-channeled region. All testing is performed in a room controlled at 23° C.±3° C. and 50%±2% relative humidity, and test samples and specimens are conditioned in this environment for at least 2 hours prior to testing.

For the channel width and channel depth measurements, three-dimensional surface topography images of the body-facing side of the test sample are recorded using an optical three-dimensional surface topography measurement system. A suitable optical three-dimensional surface topography measurement system is the MikroCAD Premium instrument commercially available from LMI Technologies Inc., Vancouver, Canada, or equivalent. The system includes the following main components: a) a Digital Light Processing (DLP) projector with direct digital controlled micro-mirrors; b) a CCD camera with at least a 1600×1200 pixel resolution; c) projection optics adapted to a measuring area of at least 140 mm×105 mm; d) recording optics adapted to a measuring area of 140 mm×105 mm; e) a table tripod based on a small hard stone plate; f) a blue LED light source; g) a measuring, control, and evaluation computer running surface texture analysis software (a suitable software is MikroCAD software with MountainsMap technology, or equivalent); and h) calibration plates for lateral (XY) and vertical (Z) calibration available from the vendor. The optical three-dimensional surface topography measurement system measures the surface height of a sample using the digital micro-mirror pattern fringe projection technique. The result of the measurement is a three-dimensional image of surface height (defined as the Z axis) versus displacement in the horizontal (XY) plane. The system has a field of view of 140×105 mm with an XY pixel resolution of approximately 85 microns. The height resolution is set to 0.5 micron/count, with a height range of +/−10 mm. Prior to testing, the instrument is calibrated according to manufacturer's specifications using the calibration plates for lateral (XY plane) and vertical (Z axis) available from the vendor.

Prepare the absorbent article test sample for surface topography measurements as follows. Unfold the absorbent article if necessary but keep the protective covering over the panty fastening adhesive (i.e. wrapper or release paper) in place. Identify and label the front and rear of the article. If the article was previously folded, use scissors or an equivalent sharp cutting device to make a cut along the width of the article at a location that is about 1 cm outboard and parallel to the rear fold line such that any residua of folded or creased material is removed. The middle and front regions of the article are discarded and the rear region is retained as the test sample with a length of about 60 mm but not less than 40 mm. With the residua of folded material removed, the test sample will lie flat against a horizontal rigid surface. Now remove the protective covering from the panty fastening adhesive and apply a light dusting of talc powder to the adhesive to mitigate tackiness. In like fashion, prepare a total of five replicate test samples.

Acquire a three-dimensional surface topography image of the test sample as follows. Transfer the test sample onto the MikroCAD (or equivalent) table beneath the camera. Orient the test sample such that the longitudinal axis 80 of the absorbent article is perpendicular to the long axis (X axis) of the instrument's field of view and the center of the flex bond channel region 160 formed along the longitudinal axis 80 is centered beneath the camera. A three-dimensional surface topography image of the test sample is collected following the instrument manufacturer's recommended measurement procedures, which may include focusing the measurement system and performing a brightness adjustment. No pre-filtering options are used. The collected height image file is saved to the evaluation computer running the surface texture analysis software.

The three-dimensional surface topography image is opened in the surface texture analysis software. The following filtering procedure is then performed on the image: 1) removal of invalid points; 2) a 3×3 pixel median filter to remove noise; and 3) a 3×3 pixel mean filter to smooth the surface.

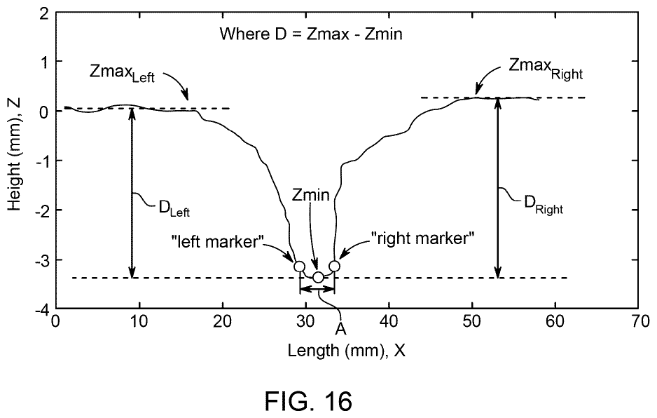

A two-dimensional line profile (a subsampling of the three-dimensional surface image) is extracted from a location within one of the individual depressed regions (within the flex bond channel region formed along the longitudinal axis 80 of the absorbent article). The line profile is perpendicular to the long side of the depression (i.e. the line traverses the width of the depression). This line profile extends across the entire width of the individual channel depression along its central lateral axis of the depression and includes non-depressed regions directly adjacent to both sides of the depression. One of skill in the art knows if the resulting line profile is not generally representative of the general contour of the flex bond channel region, owing to measurement noise or the presence of local wrinkling or a malformed channel (i.e. a channel that has a depth less than 1 mm), that another test location at a separate depressed region of the flex bond channel should be measured such that no such artifacts exist. Now create the height profile of the line (height (mm) versus line length (mm)). It will be obvious to one of skill in the art where the flex bond channel region (minimum Z value) and the non-channeled regions (maximum Z values adjacent to the channel) are located on the height profile, for example as depicted in FIG. 16.

To measure the flex bond channel width “A,” first determine the minimum height value (y axis) on the line profile (Zmin). Now move along the line on the left side of the profile in the z-direction (y axis) from the minimum height value to a position that is 200 microns greater than the minimum height value, and set a “left marker” on the line profile. In like fashion, move along the line on the right side of the profile in the z-direction (y axis) from the minimum height value to a position that is 200 microns greater than the minimum height value, and set a “right marker” on the line profile. Measure the horizontal x distance between the left and right markers placed on the line profile and record as flex bond channel width to the nearest 0.1 mm. To measure the flex bond channel depth “D,” determine the Z maximum height values (y axis) on the line profile to the left of the depression (Zmax_{Left}) and to the right of the depression (Zmax_{Right}) and record each to the nearest 0.1 mm. Calculate the difference between Zmax_{Left} and Zmin and record as D_{Left} to the nearest 0.1. Calculate the difference between Zmax_{Right} and Zmin and record as D_{Right} to the nearest 0.1 mm. Now calculate the average of D_{Left} and D_{Right} and record as flex bond channel depth to the nearest 0.1 mm.

Now repeat the entire procedure until a total of five separate, individual depressed regions of the flex bond channel region has been analyzed on the test sample. In like fashion, measure a total of five separate, individual depressed regions of the flex bond channels on each of the remaining four replicate test samples. Now calculate the arithmetic mean across all the flex bond channel width values recorded across all five test sample replicates, and report as Flex Bond Channel Width to the nearest 0.1 mm. In like fashion, calculate the arithmetic mean across all the flex bond channel depth values recorded across all five test sample replicates, and report as Flex Bond Channel Depth to the nearest 0.1 mm.

### Fiber Decitex (Dtex)

Textile webs (e.g., woven, nonwoven, airlaid) are comprised of individual fibers of material. Fibers are measured in terms of linear mass density reported in units of decitex. The decitex value is the mass in grams of a fiber present in 10,000 meters of that fiber. The decitex value of the fibers within a web of material is often reported by manufacturers as part of a specification. If the decitex value of the fiber is not known, it can be calculated by measuring the cross-sectional area of the fiber via a suitable microscopy technique such as scanning electron microscopy (SEM), determining the composition of the fiber with suitable techniques such as FTIR (Fourier Transform Infrared) spectroscopy and/or DSC (Dynamic Scanning Calorimetry), and then using a literature value for density of the composition to calculate the mass in grams of the fiber present in 10,000 meters of the fiber. All testing is performed in a room maintained at a temperature of 23° C.±2.0° C. and a relative humidity of 50%±2% and samples are conditioned under the same environmental conditions for at least 2 hours prior to testing.

If necessary, a representative sample of web material of interest can be excised from an absorbent article. In this case, the web material is removed so as not to stretch, distort, or contaminate the sample.

SEM images are obtained and analyzed as follows to determine the cross-sectional area of a fiber. To analyze the cross section of a sample of web material, a test specimen is prepared as follows. Cut a specimen from the web that is about 1.5 cm (height) by 2.5 cm (length) and free from folds or wrinkles. Submerge the specimen in liquid nitrogen and fracture an edge along the specimen's length with a razor blade (VWR Single Edge Industrial Razor blade No. 9, surgical carbon steel). Sputter coat the specimen with gold and then adhere it to an SEM mount using double-sided conductive tape (Cu, 3M available from electron microscopy sciences). The specimen is oriented such that the cross section is as perpendicular as possible to the detector to minimize any oblique distortion in the measured cross sections. An SEM image is obtained at a resolution sufficient to clearly elucidate the cross sections of the fibers present in the specimen. Fiber cross sections may vary in shape, and some fibers may consist of a plurality of individual filaments. Regardless, the area of each of the fiber cross sections is determined (for example, using diameters for round fibers, major and minor axes for elliptical fibers, and image analysis for more complicated shapes). If fiber cross sections indicate inhomogeneous cross-sectional composition, the area of each recognizable component is recorded and dtex contributions are calculated for each component and subsequently summed. For example, if the fiber is bi-component, the cross-sectional area is measured separately for the core and sheath, and dtex contribution from core and sheath are each calculated and summed. If the fiber is hollow, the cross-sectional area excludes the inner portion of the fiber comprised of air, which does not appreciably contribute to fiber dtex. Altogether, at least 100 such measurements of cross-sectional area are made for each fiber type present in the specimen, and the arithmetic mean of the cross-sectional area aₖ for each are recorded in units of micrometers squared (μm²) to the nearest 0.1 μm².

Fiber composition is determined using common characterization techniques such as FTIR spectroscopy. For more complicated fiber compositions (such as polypropylene core/polyethylene sheath bi-component fibers), a combination of common techniques (e.g., FTIR spectroscopy and DSC) may be required to fully characterize the fiber composition. Repeat this process for each fiber type present in the web material.

The decitex dₖ value for each fiber type in the web material is calculated as follows:

$${d}_{k} = 10000 m \times {a}_{k} \times {ρ}_{k} \times 1 {0}^{- 6}$$

where dₖ is in units of grams (per calculated 10,000 meter length), aₖ is in units of μm², and ρₖ is in units of grams per cubic centimeter (g/cm³). Decitex is reported to the nearest 0.1 g (per calculated 10,000 meter length) along with the fiber type (e.g., PP, PET, cellulose, PP/PET bico).

### Combinations/Examples

A. A disposable absorbent article comprising: a topsheet; a backsheet; and an absorbent core structure disposed between the topsheet and the backsheet, wherein the absorbent core structure comprises: an upper nonwoven layer; a lower nonwoven layer; and an inner core layer disposed between the upper nonwoven layer and the lower nonwoven layer, wherein the inner core layer comprises cellulosic fibers; wherein the inner core layer comprises a first absorbent zone having a first basis weight and a second absorbent zone having a second basis weight, the first basis weight is greater than the second basis weight; and wherein the first absorbent zone comprises two lateral side zones and a central zone, wherein an outer portion of the central zone is positioned inward from an outer portion of the two lateral side zones, and wherein at least a portion the central zone is continuous with at least a portion of the two lateral side zones; wherein the inner core layer is a unitary structure.

B. The disposable absorbent article of paragraph A, wherein an inner edge of the two lateral side zones and a front edge of the central zone form a continuous curve.

C. The disposable absorbent article of paragraph A or paragraph B, wherein at least one of the upper nonwoven layer and the lower nonwoven layer comprise polymer fibers.

D. The disposable absorbent article of paragraph A or paragraph B, wherein at least one of the upper nonwoven layer and the lower nonwoven layer comprise natural fibers.

E. The disposable absorbent article of any one of paragraphs A-D, wherein at least a portion of the two lateral side zones are curved.

F. The disposable absorbent article of any one of paragraphs A-E, wherein the lateral side zones are symmetric with respect to a longitudinal axis of the disposable absorbent article and asymmetric with respect to a lateral axis of the disposable absorbent article.

G. The disposable absorbent article of any one of paragraphs A-F, wherein at least a portion of the lateral side zones have a radius of curvature of from about 35 mm to about 60 mm.

H. The disposable absorbent article of any one of paragraphs A-G, wherein the two lateral side zones have a longitudinal length of from about 50 mm to about 100 mm.

I. The disposable absorbent article of any one of paragraphs A-H, wherein the central absorbent zone has a longitudinal length of from about 15 mm to about 50 mm.

J. The disposable absorbent article of any one of paragraphs A-I, wherein each of the two lateral sides comprises an outer edge, the outer edge comprising a first end and a second end, wherein the first end has a first angle with respect to a lateral axis of the disposable absorbent article and the second end has a second angle with respect to the lateral axis of the disposable absorbent article, wherein the first angle and the second angle are different.

K. The disposable absorbent article of paragraph J, wherein the first end is closer to a front edge of the disposable absorbent article and the second end is closer to a rear edge of the disposable absorbent article.

L. The disposable absorbent article of either paragraph J or paragraph K, wherein the first angle is from about 5 degrees to about 25 degrees, and the second angle is from about 30 degrees to about 60 degrees.

M. The disposable absorbent article of any one of paragraphs A-L, wherein the central zone has a width of from about 5 mm to about 20 mm.

N. The disposable absorbent article of any one of paragraphs A-M, wherein the central zone covers a lateral length of from about 12% to about 25% when measured along a latitudinal axis at the widest point of the central absorbent zone.

O. The disposable absorbent article of any one of paragraphs A-N, wherein the two lateral side zones cover from about 10% to about 25% of a longitudinal axis of the disposable absorbent article.

P. The disposable absorbent article of any one of paragraphs A-O, wherein a maximum gap between the two lateral side zones is from about 40 mm to about 70 mm.

Q. The disposable absorbent article of any one of paragraphs A-P, wherein a maximum gap between the two lateral side zones is from about 50% to about 80% a transverse axis of the inner core layer of the disposable absorbent article.

R. The disposable absorbent article of any one of paragraphs A-Q, wherein a total area of the first absorbent zone is from about 200 mm² to about 1080 mm².

S. The disposable absorbent article of any one of paragraphs A-R, wherein the central absorbent zone has a length that is from about 15% to about 50% a length of the two lateral side zones.

T. The disposable absorbent article of any one of paragraphs A-S, wherein the two lateral side zones are a distance of from about 35 mm to about 75 mm from a front edge of the disposable absorbent article.

U. The disposable absorbent article according to paragraphs A-T, wherein the inner core layer further comprises a flex bond channel.

V. The disposable absorbent article of paragraph U, wherein an inner edge of the two lateral side zones and a front edge of the central zone form a continuous curve, wherein the flex bond channel is positioned to follow the continuous curve.

W. The disposable absorbent article of paragraph U or paragraph V, wherein the flex bond channel is positioned from about 1 mm to about 10 mm forward of the continuous curve.

X. The disposable absorbent article of any one of paragraphs U-W wherein the flex bond channel region has a dry channel depth of from about 1.0 mm to about 4.5 mm and a channel width of from about 1.0 mm to about 3.0 mm.

Y. The disposable absorbent article of any one of paragraphs A-X, wherein the absorbent article has a first average density measured in the first absorbent zone and a second average density measured in the second absorbent zone, wherein the second average density is within about 0 to about 20% of the first density.

Z. The disposable absorbent article of any one of paragraphs A-Y, wherein the absorbent article has a first average density measured in the first absorbent zone and a second average density measured in the second absorbent zone, wherein the first and second average density are between about 0.045 g/cm³ and about 0.150 g/cm³.

AA. The disposable absorbent article of any one of paragraphs A-Z, wherein the upper nonwoven has a basis weight of from about 30 gsm to about 85 gsm and the lower nonwoven has a basis weight of from about 7 gsm to about 40 gsm.

BB. The disposable absorbent article of any one of paragraphs A-AA, wherein absorbent article has a caliper measured in the first absorbent zone of from about 2 mm to about 6 mm.

CC. The disposable absorbent article of any one of paragraphs A-BB, wherein the fibers of the upper nonwoven layer have a fiber diameter of from about 2.0 Dtex to about 10 Dtex and the fibers of the lower nonwoven layer have a fiber diameter of from about 1.7 Dtex to about 5 Dtex.

DD. The disposable absorbent article of any one of paragraphs A-CC, wherein the first basis weight and the second basis weight differ by about 20% to about 100%.

EE. The disposable absorbent article of any one of paragraphs A-DD, wherein the first absorbent zone has a first caliper and the second absorbent zone has a second caliper, wherein a ratio of the first caliper to the second caliper is from about 1.2 to about 2.5.

FF. The disposable absorbent article of any one of paragraphs A-EE, wherein the first basis weight is from about 180 gsm to about 450 gsm and the second basis weight is from about 150 gsm to about 320 gsm.

GG. The disposable absorbent article of any one of paragraphs A-FF further comprising a first wing portion extending laterally outward from a first longitudinal side of the disposable absorbent article, and a second wing portion extending laterally outward from a second longitudinal side of the disposable absorbent article.

HH. The disposable absorbent article of any one of paragraphs A-GG, wherein at least a portion of the two lateral size zones are positioned forward of a lateral centerline of the disposable absorbent article.

II. The disposable absorbent article of any one of paragraphs A-HH, wherein the upper nonwoven layer has a Permanent Strain of about 0.005 mm/mm to about 0.013 mm/mm.

JJ. The disposable absorbent article of any one of paragraphs A-II, wherein the inner core layer further comprises superabsorbent particles.

KK. The disposable absorbent article of any one of paragraphs A-JJ, wherein the inner core layer comprises from about 50% to about 85% cellulosic fibers, by weight of the inner core layer, and from about 15% to about 50% superabsorbent particles, by weight of the inner core layer.

LL. The disposable absorbent article of any one of paragraphs A-KK, wherein the absorbent core structure comprises a plurality of structural bond sites, wherein the structural bond sites have a bond area of from about 2 mm² to about 5 mm², and wherein the total structural bond area of the absorbent core structure is from about 1% to about 4% of the absorbent core structure as measured according to the Structural Bond Sites Pattern Spacing and Area Measurement Method.

MM. The disposable absorbent article of paragraph LL, wherein the average distance between the structural bond sites is from about 10 mm to about 32 mm as measured according to the Structural Bond Sites Pattern Spacing and Area Measurement Method.

NN. The disposable absorbent article of either paragraph LL or paragraph MM, wherein the first absorbent zone is substantially free from the structural bonds.

OO. The disposable absorbent article of any one of paragraphs A-NN, wherein the fibers of the upper nonwoven layer have a length of from about 10 mm to about 100 mm.

PP. The disposable absorbent article of any one of paragraphs A-GO, wherein the absorbent article exhibits a MD Bending Modulus measured in the first absorbent zone and the second absorbent zone of from about 0.03 N/mm² to about 0.18 N/mm².

QQ. The disposable absorbent article of any one of paragraphs A-PP, wherein the upper nonwoven layer has a Thickness at 70 g/cm² pressure of from about 0.2 mm to about 0.7 mm as measured according to the Nonwoven Thickness—Pressure Method.

RR. The disposable absorbent article of any one of paragraphs A-QQ, wherein the absorbent article exhibits an Energy of Z-Compression measured in the first absorbent zone and the second absorbent zone of from about 3.0 to about 8.0 N·mm.

SS. A disposable absorbent article comprising: a topsheet; a backsheet; and an absorbent core structure disposed between the topsheet and the backsheet, wherein the absorbent core structure comprises: an upper nonwoven layer; a lower nonwoven layer; and an inner core layer disposed between the upper nonwoven layer and the lower nonwoven layer, wherein the inner core layer comprises cellulosic fibers; wherein the inner core layer comprises a first absorbent zone having a first basis weight and a second absorbent zone having a second basis weight, wherein the first absorbent zone comprises two lateral side zones and the first basis weight is greater than the second basis weight; wherein at least a portion the central zone is continuous with at least a portion of the two lateral side zones; and wherein the absorbent article has a first average density measured in the first absorbent zone and a second average density measured in the second absorbent zone, wherein the second average density is within about 0% to about 20% of the first density.

TT. The disposable absorbent article of paragraph SS, wherein the inner core layer is a unitary structure.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A disposable absorbent article comprising:
a topsheet;
a backsheet; and
an absorbent core structure disposed between the topsheet and the backsheet,
wherein the absorbent core structure comprises:
an upper nonwoven layer;
a lower nonwoven layer; and
an inner core layer disposed between the upper nonwoven layer and the lower nonwoven layer, wherein the inner core layer comprises cellulosic fibers;
wherein the inner core layer comprises a first absorbent zone having a first basis weight and a second absorbent zone having a second basis weight, the first basis weight is greater than the second basis weight;
wherein the first absorbent zone comprises two lateral side zones and a central zone, wherein an outer portion of the central zone is positioned inward from an outer portion of the two lateral side zones, and wherein at least a portion the central zone is continuous with at least a portion of the two lateral side zones; and
wherein the inner core layer is a unitary structure.

2. The disposable absorbent article of claim 1, wherein at least a portion of the lateral side zones have a radius of curvature of from about 35 mm to about 60 mm.

3. The disposable absorbent article of claim 1, wherein at least one of the upper nonwoven layer and the lower nonwoven layer comprise at least one of polymer fibers and natural fibers.

4. The disposable absorbent article of claim 1, wherein the two lateral side zones cover from about 10% to about 25% of a longitudinal axis of the disposable absorbent article and the two lateral side zones have a longitudinal length of from about 50 mm to about 100 mm.

5. The disposable absorbent article of claim 1, wherein the central absorbent zone has a longitudinal length of from about 15 mm to about 50 mm and a width of from about 5 mm to about 20 mm.

6. The disposable absorbent article of claim 1, wherein each of the two lateral sides comprises an outer edge, the outer edge comprising a first end and a second end, wherein the first end has a first angle with respect to a lateral axis of the disposable absorbent article and the second end has a second angle with respect to the lateral axis of the disposable absorbent article, wherein the first angle and the second angle are different.

7. The disposable absorbent article of claim 6, wherein the first end is closer to a front edge of the disposable absorbent article and the second end is closer to a rear edge of the disposable absorbent article.

8. The disposable absorbent article of claim 1, wherein the central zone covers a lateral length of from about 12% to about 25% when measured along a latitudinal axis at the widest portion of the central absorbent zone.

9. The disposable absorbent article of claim 1, wherein a maximum gap between the two lateral side zones is from about 40 mm to about 70 mm and the maximum gap between the two lateral side zones is from about 50% to about 80% a transverse axis of the inner core layer of the disposable absorbent article.

10. The disposable absorbent article of claim 1, wherein the central absorbent zone has a length that is from about 15% to about 50% a length of the two lateral side zones.

11. The disposable absorbent article of claim 1, wherein the two lateral side zones are a distance of from about 35 mm to about 75 mm from a front edge of the disposable absorbent article.

12. The disposable absorbent article of claim 1, wherein the inner core layer further comprises a flex bond channel.

13. The disposable absorbent article of claim 12, wherein an inner edge of the two lateral side zones and a front edge of the central zone form a continuous curve, wherein the flex bond channel is positioned to follow the continuous curve from about 1 mm to about 10 mm mm forward of the continuous curve.

14. The disposable absorbent article of claim 1, wherein the absorbent article has a first average density measured in the first absorbent zone and a second average density measured in the second absorbent zone, wherein the second average density is within about 0 to about 20% of the first density.

15. The disposable absorbent article of claim 1, wherein the absorbent article has a first average density measured in the first absorbent zone and a second average density measured in the second absorbent zone, wherein the first and second average density are between about 0.045 g/cm³ and about 0.150 g/cm³.

16. The disposable absorbent article of claim 1 further comprising a first wing portion extending laterally outward from a first longitudinal side of the disposable absorbent article, and a second wing portion extending laterally outward from a second longitudinal side of the disposable absorbent article.

17. The disposable absorbent article of claim 1, wherein the inner core layer further comprises superabsorbent particles.

18. The disposable absorbent article of claim 1, wherein the absorbent core structure comprises a plurality of structural bond sites, wherein the first absorbent zone is substantially free from the structural bonds.

19. The disposable absorbent article of claim 1, wherein the absorbent article exhibits a MD Bending Modulus measured in the first absorbent zone and the second absorbent zone of from about 0.03 N/mm² to about 0.18 N/mm².

20. A disposable absorbent article comprising:
a topsheet;
a backsheet; and
an absorbent core structure disposed between the topsheet and the backsheet,
wherein the absorbent core structure comprises:
an upper nonwoven layer;
a lower nonwoven layer; and
an inner core layer disposed between the upper nonwoven layer and the lower nonwoven layer, wherein the inner core layer comprises cellulosic fibers;
wherein the inner core layer comprises a first absorbent zone having a first basis weight and a second absorbent zone having a second basis weight, wherein the first absorbent zone comprises two lateral side zones and the first basis weight is greater than the second basis weight;
wherein at least a portion the central zone is continuous with at least a portion of the two lateral side zones; and
wherein the absorbent article has a first average density measured in the first absorbent zone and a second average density measured in the second absorbent zone, wherein the second average density is within about 0% to about 20% of the first density.
